(19) United States
(12) Patent Application Publication
(10) Pub. No.: US 2024/0150441 A1
(43) Pub. Date: May 9, 2024

(54) MONOCLONAL ANTIBODIES AGAINST SARS-COV-2 AND VARIANTS

(71) Applicants: Qiang Chen, Chandler, AZ (US); Haiyan Sun, Chandler, AZ (US); Collin Jugler, Mesa, AZ (US)

(72) Inventors: Qiang Chen, Chandler, AZ (US); Haiyan Sun, Chandler, AZ (US); Collin Jugler, Mesa, AZ (US)

(73) Assignee: Arizona Board of Regents on Behalf of Arizona State University, Temep, AZ (US)

(21) Appl. No.: 18/500,684

(22) Filed: Nov. 2, 2023

Related U.S. Application Data
(60) Provisional application No. 63/422,238, filed on Nov. 3, 2022.

(51) Int. Cl. C07K 16/10; A01H 6/82; A61P 31/14; C12N 15/63

(52) U.S. Cl. CPC C07K 16/1003; A01H 6/823; A61P 31/14; C12N 15/63; C07K 2317/14

(57) ABSTRACT

The present invention provides monoclonal antibodies against SARS-CoV-2 and methods of use and making thereof.

### CROSS-REFERENCE TO RELATED APPLICATIONS

This application is a U.S. Non-Provisional application that claims benefit to U.S. Provisional Application No. 63/422,238 filed on Nov. 3, 2022, which is incorporated by reference in its entirety.

### SEQUENCE LISTING

The present application contains a Sequence Listing which has been submitted electronically in .XML format and is hereby incorporated herein by reference in its entirety. Said computer readable file, was created on Oct. 23, 2023, is named 055743_776666_SequenceListing.xml and is 53.1 kilobytes in size.

### FIELD OF THE INVENTION

The present invention relates generally to monoclonal antibodies against SARS-CoV-2 and methods of their use.

### BACKGROUND OF THE INVENTION

Coronaviruses, members of the family Coronaviridae and subfamily Coronavirinae, are enveloped viruses containing single-strand, positive-sense RNA genome ranging from 26 to 32 kilobases in length. Coronaviruses have been identified in several vertebrate hosts including bird, bat, pig, rodent, camel, and human. Human can acquire coronavirus infection from other host of mammals, which may cause detrimental upper respiratory tract illness.

Members of the coronavirus family include virus strains having different phylogenetic origin and causing different severity in mortality and morbidity. As such, treatment and prevention for coronavirus infection varies depending on the specific strains that cause the infection. Currently, we lack effective methods for prevention of coronavirus infection, and antiviral drug treatment once coronavirus infection has occurred. Accordingly, a need exists for the treatment and prevention of coronavirus infection, particularly of infection caused by SARS-CoV-2, and COVID-19 disease.

Severe acute respiratory syndrome coronavirus 2 (SARS-CoV-2) infections continue to persist around the globe, amid ongoing emergences of variants that are immune evasive. This is often attributed to mutations in the viral spike (S) protein that allow for reduced or abolished binding of neutralizing antibodies. Furthermore, most monoclonal antibodies (mAbs) that have been developed against SARS-CoV-2 infection have also lost utility against the Omicron variant and its subvariants. A continued need exists for effective therapies for SARS-CoV-2.

### SUMMARY OF THE INVENTION

In various aspects, the present disclosure encompasses a monoclonal antibody comprising a human IgG1, IgG2, IgG3 or IgG4 heavy chain backbone amino acid sequence fused to at least one of (i) an amino acid sequence of a heavy chain variable region of tixagevimab; (ii) an amino acid sequence of a heavy chain variable region of cilgavimab; and a human kappa or lambda light chain backbone fused to at least one of (iii) an amino acid sequence of a light chain variable region of tixagevimab, or (iv) an amino acid sequence of a light chain variable region of cilgavimab; wherein items (i)-(iv) comprises an Fc receptor binding domain (FcγR domain).

In another aspect, the human IgG backbone amino heavy chain backbone amino acid sequence has at least 70%, at least 80% or at least 90% sequence identity to the amino acid sequence of any one of SEQ ID NOS: 1-4 and/or wherein the human kappa or lambda light chain backbone amino acid sequence has at least 70%, at least 80%, or at least 90% sequence identity to the amino acid sequence of SEQ ID NO: 5 or 6.

In yet another aspect, the monoclonal antibody comprises a light chain variable region of tixagevimab having at least 70%, at least 80% or at least 90% sequence identity to SEQ ID NO: 10 or 11 and/comprises a heavy chain variable region of tixagevimab having at least 70%, at least 80%, or at least 90% sequence identity to SEQ ID NO: 8 or 9.

In another aspect, the monoclonal antibody comprises the amino acid sequence of the light chain variable region of cilgavimab having at least 70%, at least 80% or at least 90% sequence identity to SEQ ID NO: 14 or 15 and/or comprises the amino acid sequence of the heavy chain variable region of cilgavimab having at least 70%, at least 80%, or at least 90% sequence identity to SEQ ID NO: 12 or 13.

In another aspect, the monoclonal antibody comprises a heavy chain having an amino acid sequence of SEQ ID NO: 20 or 21 and a light chain having an amino acid sequence of SEQ ID NO: 16 or 17. In another aspect, the monoclonal antibody comprises a heavy chain having an amino acid sequence of SEQ ID NO: 22 or 23 and a light chain having the amino acid sequence of SEQ ID NO. 18 or 19. In yet another aspect, the monoclonal antibody comprises a human-like biantennary GnGn or hybrid MGn glycoform.

In one aspect, the present disclosure encompasses a nucleic acid construct encoding the above monoclonal antibody, the construct comprising a nucleic acid sequence having at least 70%, at least 80% or at least 90% sequence identity with any of SEQ. ID Nos. 35-41. In another aspect, the present disclosure encompasses a vector comprising the above nucleic acid sequence.

In one aspect, the present disclosure encompasses a transgenic Nicotiana plant comprising the above monoclonal antibody, the nucleic acid sequence thereof, and/or the vector thereof. In another aspect, the transgenic Nicotiana plant is a transgenic Nicotiana benthamiana plant. In another aspect, the transgenic Nicotiana plant has a reduced expression of β 1,2-xylosyltransferase (XylT) and/or a 1,3-fucosyltransferase (FucT). In yet another aspect, the present disclosure encompasses a monoclonal antibody produced in the above transgenic Nicotiana benthamiana plant, having reduced expression of xylosyltransferase and/or fucosyltransferase.

In one aspect, the present disclosure encompasses a pharmaceutical composition comprising any one of the above monoclonal antibodies, and any combination thereof, and further comprising a pharmaceutically acceptable carrier, diluent or excipient.

In one aspect, the present disclosure encompasses a method of making a SARS-CoV-2 mAb variant from a parent SARS-CoV-2 mAb produced in a mammalian cell, comprising the steps of: (i) fusing a nucleic acid sequence encoding a heavy chain variable region of the parent SARS-CoV-2 mAb to a nucleic acid sequence encoding a heavy chain IgG backbone, and a nucleic acid sequence encoding a light chain variable region of the parent SARS-CoV-2 mAb to a nucleic acid sequence encoding a light chain backbone; (ii) restoring a nucleic acid sequence encoding an FcγR domain to the heavy chain variable region and/or the light chain variable region of the parent SARS-CoV-2 mAb to form an expression construct for expressing the SARS-CoV-2 mAb variant; (iii) introducing the expression construct into a plant cell; and (iv) maintaining the plant cell for a time and under conditions sufficient for the plant to express the SARS-CoV-2 mAb variant. In one aspect, the method comprises the parent SARS-CoV-2 mAb selected from tixagevimab and cilgavimab. In another aspect, the method comprises the plant being a N. benthamiana plant having reduced expression of xylosyltransferase and/or fucosyltransferase, and optionally is a ΔXFT N. benthamiana plant.

In one aspect, the present disclosure encompasses a method of treating a coronavirus infection or suspected coronavirus infection, comprising administering to a subject in need thereof an effective amount of a SARS-CoV-2 mAb variant produced in a plant cell and derived from a parent SARS-CoV-2 mAb produced in a mammalian cell. In one aspect, the subject has or is suspected of having an infection by a SARS virus, SARS-CoV, SARS-CoV-2, Middle East respiratory syndrome (MERS), any variant or sub-variant of any one thereof, or any combination thereof. In another aspect, the subject is selected from a human, a non-human primate, a dog, a cat, a cow, a pig, a sheep, a chicken, a goose and a duck.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B show temporal expression of pCilgavimab and pTixagevimab in ΔXFT N. benthamiana plants. Data shown is in micrograms of mAb per gram of fresh leaf weight (FLW). DPI is days post agroinfiltration.

FIGS. 2A-2B show neutralization analysis by foci-forming assay for pCilgavimab and pTixagevimab against the live B.1.1.529 (Omicron) variant of SARS-CoV-2. The half maximal inhibitory concentration (IC50) value for each curve is indicated in the graph. Error bars are standard deviation and represent two independent experiments performed in triplicate.

FIG. 3 depicts mass spectrometry analysis of pCilgavimab and pTixagevimab glycosylation. Proportions of each glycan are represented as a percent of the total glycosylated population of mAb. GnGn, biantennary N-actelyglucosamine; MGn, monoantennary N-acetylglucosamine; Man9/8/7, mixed branching oligomannose.

FIG. 4 depicts neutralizing synergy of the pCilgavimab and pTixagevimab cocktail. HSA, highest single agent; ZIP, zero interaction potency; IC20, IC25, and IC50: 20%, 25%, and 50% inhibitory concentration, respectively. The predicated neutralization values were calculated from the neutralization data of each individual mAb in the cocktail and represent percent neutralization where there is no synergistic interaction between different mAbs.

FIG. 5A-5B depict neutralization curves generated from a luciferase reporter assay for pCilgavimab and pTixagevimab against a BA.4/5 SARS-Cov-2 pseudovirus. IC50 value for each curve is indicated.

FIG. 6 shows percent neutralization of the combination of pCilgavimab and pTixagevimab at their respective IC50 concentrations against BA.4/5 SARS-CoV-2 pseudovirus. Both the observed and predicted percent neutralization frohashe HSA and Loewe synergy models are presented. Error bars in all graphs are standard deviation and represent at least two independent experiments performed in at least duplicate.

FIG. 7 shows ADCC activity measured in ADCC bioassay reporter cells coincubated with 293-SARS2-S-dfur cells, with surface-display of the spike protein of SARS-CoV-2, in the presence of plant-produced (Green) or mammalian cell produced (red) mAb. Luciferase activity was detected using Bio-Glo reagent and the data were normalized and fitted to a response curve using GraphPad Prism 9.0. Error bars represent SD and the data were collected from at least two independent experiments with triplicates.

### DETAILED DESCRIPTION

Monoclonal antibodies are often the first in line therapy for SARS-CoV-2 infection, but many have lost utility against the Omicron variant and its subvariants. One mAb cocktail, Evusheld, retains neutralizing capacity against currently circulating variants of Omicron and is composed of two mAbs, named tixagevimab (parental mAb is COV2-2196) and cilgavimab (parental mAb is COV2-2130). These mAbs have non-overlapping epitopes on the receptor binding domain (RBD) or the S protein, making them an effective combination therapy. Disclosed herein are new antibody versions of tixagevimab and cilgavimab expressed in Nicotiana benthamiana plants. Exemplary data herein shows that the plant-made counterparts of these mAbs efficiently neutralize multiple omicron subvariants of SARS-CoV-2 including the BA.5 with the potential of enhancing their effector function.

Accordingly, in various aspects, monoclonal antibodies are provided, wherein the monoclonal antibodies comprise a human IgG backbone (e.g., IgG1, IgG2, IgG3 or IgG4) fused to at least one of (i) a variable region of tixagevimab and/or(ii) a variable region of cilgavimab, wherein each of the variable regions of tixagevimab and cilgavimab comprise an Fc receptor binding domain (FcγR). In particular aspects, the present disclosure is based on the discovery that these useful monoclonal antibodies may be successfully produced in plants with comparably human glycoforms. Accordingly, provided herein are antibodies and transgenic plants comprising them, and methods of making thereof, pharmaceutical compositions comprising the antibodies herein, and methods of administering antibodies and/or compositions herein to treat or prevent SARS-CoV-2 infection in a subject. Monoclonal Antibodies against Severe Acute Respiratory Syndrome Related Coronavirus (SARS-CoV)

The present disclosure provides antibodies, produced in genetically engineered plants, that can effectively neutralize SARS-CoV-2 virus. In particular aspects, the monoclonal antibodies described herein can neutralize omicron variants of SARS-CoV-2.

As noted, the monoclonal antibodies of the present disclosure comprise an IgG backbone and at least one variable region from tixagevimab or cilgavimab, wherein each variable region further comprises an Fc receptor binding domain (FcγR domain).

The IgG backbone may comprise an IgG1, an IgG2, an IgG3 or an IgG4 backbone and may, optionally, comprise an amino acid sequence having at least 70%, at least 80%, or at least 90% sequence identity to the amino acid sequence of any one of SEQ ID NOs: 1-4. In some aspects, the monoclonal antibody may comprise an IgG1 backbone comprising an amino acid sequence having at least about 70%, at least about 75%, at least about 80%, at least about 85%, at least about 90%, at least about 95% at least about 98% or at least about 99% sequence identity to SEQ ID NO: 1. In some aspects, the monoclonal antibody may comprise an IgG2 backbone comprising an amino acid sequence having at least about 70%, at least about 75%, at least about 80%, at least about 85%, at least about 90%, at least about 95% at least about 98% or at least about 99% sequence identity to SEQ ID NO: 2. In some aspects, the monoclonal antibody may comprise an IgG3 backbone comprising an amino acid sequence having at least about 70%, at least about 75%, at least about 80%, at least about 85%, at least about 90%, at least about 95% at least about 98% or at least about 99% sequence identity to SEQ ID NO: 3. In some aspects, the monoclonal antibody may comprise an IgG4 backbone comprising an amino acid sequence having at least about 70%, at least about 75%, at least about 80%, at least about 85%, at least about 90%, at least about 95% at least about 98% or at least about 99% sequence identity to SEQ ID NO: 4. In various aspects, the monoclonal antibody may comprise an IgG backbone having an amino acid sequence comprising or consisting of any one of SEQ ID NOs: 1-4. For ease of reference, SEQ ID NOs 1˜4 are provided in Table 1 below.

TABLE 1

| | | SEQ |
|---|---|---|
| Descrip- | | ID |
| tion | Sequence | NO: |
| Human | ASTKGPSVFPLAPSSKSTSGGTAALGCLVKDY | 1 |
| IgG1 | FPEPVTVSWNSGALTSGVHTFPAVLQSSGLYS | |
| Heavy | LSSVVTVPSSSLGTQTYICNVNHKPSNTKVDKK | |
| chain | VEPKSCDKTHTCPPCPAPELLGGPSVFLFPPK | |
| | PKDTLMISRTPEVTCVVVDVSHEDPEVKFNWY | |
| | VDGVEVHNAKTKPREEQYNSTYRVVSVLTVLH | |
| | QDWLNGKEYKCKVSNKALPAPIEKTISKAKGQ | |
| | PREPQVYTLPPSRDELTKNQVSLTCLVKGFYP | |
| | SDIAVEWESNGQPENNYKTTPPVLDSDGSFFL | |
| | YSKLTVDKSRWQQGNVFSCSVMHEALHNHYT | |
| | QKSLSLSPGK | |
| Human | ASTKGPSVFPLAPCSRSTSESTAALGCLVKDYF | 2 |
| IgG2 | PEPVTVSWNSGALTSGVHTFPAVLQSSGLYSL | |
| Heavy | SSVVTVPSSNFGTQTYTCNVDHKPSNTKVDKT | |
| chain | VERKCCVECPPCPAPPVAGPSVFLFPPKPKDT | |
| | LMISRTPEVTCVVDVSHEDPEVQFNWYVDGV | |
| | EVHNAKTKPREEQFNSTFRVVSVLTVVHQDWL | |
| | NGKEYKCKVSNKGLPAPIEKTISKTKGQPREPQ | |
| | VYTLPPSREEMTKNQVSLTCLVKGFYPSDISVE | |
| | WESNGQPENNYKTTPPMLDSDGSFFLYSKLTV | |
| | DKSRWQQGNVFSCSVMHEALHNHYTQKSLSL | |
| | SPGK | |
| Human | ASTKGPSVFPLAPCSRSTSGGTAALGCLVKDY | 3 |
| IgG3 | FPEPVTVSWNSGALTSGVHTFPAVLQSSGLYS | |
| Heavy | LSSVVTVPSSSLGTQTYTCNVNHKPSNTKVDK | |
| chain | RVELKTPLGDTTHTCPRCPEPKSCDTPPPCPR | |
| | CPEPKSCDTPPPCPRCPEPKSCDTPPPCPRCP | |
| | APELLGGPSVFLFPPKPKDTLMISRTPEVTCWV | |
| | VDVSHEDPEVQFKWYVDGVEVHNAKTKPREE | |
| | QYNSTFRVVSVLTVLHQDWLNGKEYKCKVSNK | |
| | ALPAPIEKTISKTKGQPREPQVYTLPPSREEMT | |
| | KNQVSLTCLVKGFYPSDIAVEWESSGQPENNY | |
| | NTTPPMLDSDGSFFLYSKLTVDKSRWQQGNIF | |
| | SCSVMHEALHNRFTQKSLSLSPGK | |
| Human | ASTKGPSVFPLAPCSRSTSESTAALGCLVKDYF | 4 |
| IgG4 | PEPVTVSWNSGALTSGVHTFPAVLQSSGLYSL | |
| Heavyhain | SSVVTVPSSSLGTKTYTCNVDHKPSNTKVDKR | |
| | VESKYGPPCPSCPAPEFLGGPSVFLFPPKPKD | |
| | TLMISRTPEVTCVWVDVSQEDPEVQFNWYVDG | |
| | VEVHNAKTKPREEQFNSTYRVVSVLTVLHQDW | |
| | LNGKEYKCKVSNKGLPSSIEKTISKAKGQPREP | |
| | QVYTLPPSQEEMTKNQVSLTCLVKGFYPSDIAV | |
| | EWESNGQPENNYKTTPPVLDSDGSFFLYSRLT | |
| | VDKSRWQEGNVFSCSVMHEALHNHYTQKSLS | |
| | LSLGK | |
| Human | RTVAAPSVFIFPPSDEQLKSGTASVVCLLNNFY | 5 |
| Kappa | PREAKVQWKVDNALQSGNSQESVTEQDSKDS | |
| light | TYSLSSTLTLSKADYEKHKVYACEVTHQGLSSP | |
| chain | VTKSFNRGEC | |
| Human | GQPKANPTVTLFPPSSEELQANKATLVCLISDF | 6 |
| Lambda | YPGAVTVAWKADGSPVKAGVETTKPSKQSNN | |
| light | KYAASSYLSLTPEQWKSHRSYSCQVTHEGSTV | |
| chain | EKTVAPTECS | |

In various aspects, the monoclonal antibody may comprise one or more heavy or light chain variable regions derived from heavy chain variable region or light chain variable region of Tixagevimab or Cilgavimab. As is understood in the art, antibodies prepared in a heterologous expression system (such as the plant species described herein) are often prepared with an appropriate signal peptide at the N-terminus. In some aspects, the signal peptide is a plant signal peptide. In further aspects, the signal peptide comprises or consists of SEQ ID NO: 7 (MGWSCIILFLVATATGVHS). All of the variable regions described herein (e.g., any of the heavy chain variable regions and/or the light chain variable regions) and any of the monoclonal antibodies described herein may optionally comprise a suitable signal peptide (e.g., SEQ ID NO: 7, MGWSCIILFLVATATGVHS) at the N-terminus.

In various aspects, the monoclonal antibodies may comprise a heavy chain variable region having at least 70%, at least 80%, at least 90% sequence identity to the heavy chain variable region of Tixagevimab (e.g., SEQ ID NO: 8). In some aspects, the monoclonal antibodies may comprise a heavy chain variable region comprising at least about 70%, at least about 75%, at least about 80%, at least about 85%, at least about 90%, at least about 95% at least about 98% or at least about 99% sequence identity to SEQ ID NO: 8. For example, in some aspects, the amino acid sequence of the heavy chain variable region may comprise at least 70% sequence identity to SEQ ID NO: 8. For example, in some aspects, the amino acid sequence of the heavy chain variable region may comprise at least 75% sequence identity to SEQ ID NO: 8. For example, in some aspects, the amino acid sequence of the heavy chain variable region may comprise at least 80% sequence identity to SEQ ID NO: 8. For example, in some aspects, the amino acid sequence of the heavy chain variable region may comprise at least 85% sequence identity to SEQ ID NO: 8. For example, in some aspects, the amino acid sequence of the heavy chain variable region may comprise at least 90% sequence identity to SEQ ID NO: 8. For example, in some aspects, the amino acid sequence of the heavy chain variable region may comprise at least 95% sequence identity to SEQ ID NO: 8. For example, in some aspects, the amino acid sequence of the heavy chain variable region may comprise at least 98% sequence identity to SEQ ID NO: 8. For example, in some aspects, the amino acid sequence of the heavy chain variable region may comprise at least 99% sequence identity to SEQ ID NO: 8. In some aspects, the amino acid sequence of the heavy chain variable region comprises or consists of SEQ ID NO: 8.

In various aspects, the monoclonal antibodies may comprise a heavy chain variable region having at least 70%, at least 80%, at least 90% sequence identity to the heavy chain variable region of cilgavimab (e.g., SEQ ID NO: 12). In some aspects, the monoclonal antibodies may comprise a heavy chain variable region comprising at least about 70%, at least about 75%, at least about 80%, at least about 85%, at least about 90%, at least about 95% at least about 98% or at least about 99% sequence identity to SEQ ID NO: 12. For example, in some aspects, the amino acid sequence of the heavy chain variable region may comprise at least 70% sequence identity to SEQ ID NO: 12. For example, in some aspects, the amino acid sequence of the heavy chain variable region may comprise at least 75% sequence identity to SEQ ID NO: 12. For example, in some aspects, the amino acid sequence of the heavy chain variable region may comprise at least 80% sequence identity to SEQ ID NO: 12. For example, in some aspects, the amino acid sequence of the heavy chain variable region may comprise at least 85% sequence identity to SEQ ID NO: 12. For example, in some aspects, the amino acid sequence of the heavy chain variable region may comprise at least 90% sequence identity to SEQ ID NO: 12. For example, in some aspects, the amino acid sequence of the heavy chain variable region may comprise at least 95% sequence identity to SEQ ID NO: 12. For example, in some aspects, the amino acid sequence of the heavy chain variable region may comprise at least 98% sequence identity to SEQ ID NO: 12. For example, in some aspects, the amino acid sequence of the heavy chain variable region may comprise at least 99% sequence identity to SEQ ID NO: 12. In some aspects, the amino acid sequence of the heavy chain variable region comprises or consists of SEQ ID NO: 12.

In various aspects, the monoclonal antibodies may comprise a light chain variable region having at least 70%, at least 80%, at least 90% sequence identity to the light chain variable region of tixagevimab (e.g., SEQ ID NO: 10). In some aspects, the monoclonal antibodies may comprise a light chain variable region comprising at least about 70%, at least about 75%, at least about 80%, at least about 85%, at least about 90%, at least about 95% at least about 98% or at least about 99% sequence identity to SEQ ID NO: 10. For example, in some aspects, the amino acid sequence of the light chain variable region may comprise at least 70% sequence identity to SEQ ID NO: 10. For example, in some aspects, the amino acid sequence of the light chain variable region may comprise at least 75% sequence identity to SEQ ID NO: 10. For example, in some aspects, the amino acid sequence of the light chain variable region may comprise at least 80% sequence identity to SEQ ID NO: 10. For example, in some aspects, the amino acid sequence of the light chain variable region may comprise at least 85% sequence identity to SEQ ID NO: 10. For example, in some aspects, the amino acid sequence of the light chain variable region may comprise at least 90% sequence identity to SEQ ID NO: 10. For example, in some aspects, the amino acid sequence of the light chain variable region may comprise at least 95% sequence identity to SEQ ID NO: 10. For example, in some aspects, the amino acid sequence of the light chain variable region may comprise at least 98% sequence identity to SEQ ID NO: 10. For example, in some aspects, the amino acid sequence of the light chain variable region may comprise at least 99% sequence identity to SEQ ID NO: 10. In some aspects, the amino acid sequence of the light chain variable region comprises or consists of SEQ ID NO: 10.

In various aspects, the monoclonal antibodies may comprise a light chain variable region having at least 70%, at least 80%, at least 90% sequence identity to the light chain variable region of cilgavimab (e.g., SEQ ID NO: 14). In some aspects, the monoclonal antibodies may comprise a light chain variable region comprising at least about 70%, at least about 75%, at least about 80%, at least about 85%, at least about 90%, at least about 95% at least about 98% or at least about 99% sequence identity to SEQ ID NO: 14. For example, in some aspects, the amino acid sequence of the light chain variable region may comprise at least 70% sequence identity to SEQ ID NO: 14. For example, in some aspects, the amino acid sequence of the light chain variable region may comprise at least 75% sequence identity to SEQ ID NO: 14. For example, in some aspects, the amino acid sequence of the light chain variable region may comprise at least 80% sequence identity to SEQ ID NO: 14. For example, in some aspects, the amino acid sequence of the light chain variable region may comprise at least 85% sequence identity to SEQ ID NO: 14. For example, in some aspects, the amino acid sequence of the light chain variable region may comprise at least 90% sequence identity to SEQ ID NO: 14. For example, in some aspects, the amino acid sequence of the light chain variable region may comprise at least 95% sequence identity to SEQ ID NO: 14. For example, in some aspects, the amino acid sequence of the light chain variable region may comprise at least 98% sequence identity to SEQ ID NO: 14. For example, in some aspects, the amino acid sequence of the light chain variable region may comprise at least 99% sequence identity to SEQ ID NO: 14. In some aspects, the amino acid sequence of the light chain variable region comprises or consists of SEQ ID NO: 14.

As mentioned, any of the variable regions of the antibodies described herein may further comprise a signal peptide (e.g., SEQ ID NO: 7, MGWSCIILFLVATATGVHS). When the signal peptide is included in the heavy chain variable region of tixagevimab, the resulting variable region comprises or consists of SEQ ID NO: 9. Accordingly, in various aspects, the monoclonal antibody may comprise a heavy chain variable region having at least 70%, at least 75%, at least 80%, at least 85%, at least 90%, at least 95%, at least 98%, or at least 99% sequence identity to SEQ ID NO: 9. When the signal peptide is included in the heavy chain variable region of cilgavimab, the resulting variable region comprises or consists of SEQ ID NO: 13. Accordingly, in various aspects, the monoclonal antibody may comprise a heavy chain variable region having at least 70%, at least 75%, at least 80%, at least 85%, at least 90%, at least 95%, at least 98%, or at least 99% sequence identity to SEQ ID NO: 13. When the signal peptide is included in the light chain variable region of tixagevimab, the resulting variable region comprises or consists of SEQ ID NO: 11. Accordingly, in various aspects, the monoclonal antibody may comprise a light chain variable region having at least 70%, at least 75%, at least 80%, at least 85%, at least 90%, at least 95%, at least 98%, or at least 99% sequence identity to SEQ ID NO: 11. When the signal peptide is included in the light chain variable region of cilgavimab, the resulting variable region comprises or consists of SEQ ID NO: 15. Accordingly, in various aspects, the monoclonal antibody may comprise a light chain variable region having at least 70%, at least 75%, at least 80%, at least 85%, at least 90%, at least 95%, at least 98%, or at least 99% sequence identity to SEQ ID NO: 15.

For ease of reference, exemplary sequences described above are included in Table 2 below.

TABLE 2

| | | SEQ |
|---|---|---|
| | | ID |
| Description | Sequence | NO: |
| Tixagevimab heavy chain | QMQLVQSGPEVKKPGTSVKVSCKASGFTFMSS | 8 |
| variable region (no signal | AVQWVRQARGQRLEWIGWIVIGSGNTNYAQKF | |
| peptide) | QERVTITRDMSTSTAYMELSSLRSEDTAVYYC | |
| | AAPYCSSISCNDGFDIWGQGTMVTVSS | |
| Tixagevimab heavy chain | MGWSCIILFLVATATGVHSQMQLVQSGPEVKK | 9 |
| variable region with signal | PGTSVKVSCKASGFTFMSSAVQWVRQARGQRL | |
| peptide (underlined and | EWIGWIVIGSGNTNYAQKFQERVTITRDMSTS | |
| bolded) | TAYMELSSLRSEDTAVYYCAAPYCSSISCNDG | |
| | FDIWGQGTMVTVSS | |
| Tixagevimab light chain | EIVLTQSPGTLSLSPGERATLSCRASQSVSSSY | 10 |
| variable region (no signal | LAWYQQKPGQAPRLLIYGASSRATGIPDRFSGS | |
| peptide) | GSGTDFTLTISRLEPEDFAVYYCQHYGSSRGWT | |
| | FGQGTKVEIK | |
| Tixagevimab light chain | MGWSCIILFLVATATGVHSEIVLTQSPGTLSLS | 11 |
| variable region with signal | PGERATLSCRASQSVSSSYLAWYQQKPGQAPRL | |
| peptide (underlined and | LIYGASSRATGIPDRFSGSGSGTDFTLTISRLE | |
| bolded) | PEDFAVYYCQHYGSSRGWTFGQGTKVEIK | |
| Cilgavimab heavy chain | EVQLVESGGGLVKPGGSLRLSCAASGFTFRDV | 12 |
| variable region (no signal | WMSWVRQAPGKGLEWVGRIKSKIDGGTTDYAA | |
| peptide) | PVKGRFTISRDDSKNTLYLQMNSLKTEDTAVY | |
| | YCTTAGSYYYDTVGPGLPEGKFDYWGQGTLVT | |
| | VSS | |
| Cilgavimab heavy chain | MGWSCIILFLVATATGVHSEVQLVESGGGLVK | 13 |
| variable region with signal | PGGSLRLSCAASGFTFRDVWMSWVRQAPGKGL | |
| peptide (underlined and | EWVGRIKSKIDGGTTDYAAPVKGRFTISRDDS | |
| bolded) | KNTLYLQMNSLKTEDTAVYYCTTAGSYYYDTV | |
| | GPGLPEGKFDYWGQGTLVTVSS | |
| Cilgavimab light chain variable | DIVMTQSPDSLAVSLGERATINCKSSQSVLYS | |
| region (no signal peptide) | SNNKNYLAWYQQKPGQPPKLLMYWASTRESGV | 14 |
| | PDRFSGSGSGAEFTLTISSLQAEDVAIYYCQQ | |
| | YYSTLTFGGGTKVEIK | |
| Cilgavimab light chain variable | MGWSCIILFLVATATGVHSDIVMTQSPDSLAVS | 15 |
| region with signal peptide | LGERATINCKSSQSVLYSSNNKNYLAWYQQKPG | |
| (underlined and bolded) | QPPKLLMYWASTRESGVPDRFSGSGSGAEFTLT | |
| | ISSLQAEDVAIYYCQQYYSTLTFGGGTKVEIK | |

In various aspects, the monoclonal antibodies may comprise a light chain comprising an amino acid sequence of SEQ ID NO: 16 (or comprising an amino acid sequence having at least 70%, at least 75%, at least 80%, at least 85%, at least 90%, at least 95%, at least 98%, at least 99% sequence identity to SEQ ID NO: 16). In various aspects, the monoclonal antibody may comprise a light chain comprising SEQ ID NO: 18 (or comprising an amino acid sequence having at least 70%, at least 75%, at least 80%, at least 85%, at least 90%, at least 95%, at least 98%, at least 99% sequence identity to SEQ ID NO: 18). SEQ ID NOs 16 and 18 do not comprise the optional signal peptide described above. In some aspects, the signal peptide may be included and in these aspects the monoclonal antibody may comprise a light chain comprising or consist of an amino acid sequence of SEQ ID NO: 17 or SEQ ID NO: 19 (or an amino acid sequence having at least 70%, at least 75%, at least 80%, at least 85%, at least 90%, at least 95%, at least 98%, at least 99% sequence identity to SEQ ID NO: 17 or 19. For ease of references, SEQ ID NOs: 16 to 19 are provided in Table 3 below.

TABLE 3

| | | SEQ |
|---|---|---|
| | | ID |
| Description | Sequence | NO: |
| Full light chain of | EIVLTQSPGTLSLSPGERATLSCRASQSVSSSYLAWY | 16 |
| Tixagevimab (no signal | QQKPGQAPRLLIYGASSRATGIPDRFSGSGSGTDFTL | |
| peptide) | TISRLEPEDFAVYYCQHYGSSRGWTFGQGTKVEIKR | |
| | TVAAPSVFIFPPSDEQLKSGTASWVCLLNNFYPREAK | |
| | VQWKVDNALQSGNSQESVTEQDSKDSTYSLSSTLTL | |
| | SKADYEKHKVYACEVTHQGLSSPVTKSFNRGEC | |
| Full light chain of | MGWSCIILFLVATATGVHSEIVLTQSPGTLSLSPGER | 17 |
| Tixagevimab (with signal | ATLSCRASQSVSSSYLAWYQQKPGQAPRLLIYGASS | |
| peptide underlined and | RATGIPDRFSGSGSGTDFTLTISRLEPEDFAVYYCQH | |
| bolded) | YGSSRGWTFGQGTKVEIKRTVAAPSVFIFPPSDEQLK | |
| | SGTASVVCLLNNFYPREAKVQWKVDNALQSGNSQE | |
| | SVTEQDSKDSTYSLSSTLTLSKADYEKHKVYACEVTH | |
| | QGLSSPVTKSFNRGEC | |
| Full light chain of | DIVMTQSPDSLAVSLGERATINCKSSQSVLYSSNNKN | 18 |
| Cilgavimab (no signal | YLAWYQQKPGQPPKLLMYWASTRESGVPDRFSGSGS | |
| peptide) | GAEFTLTISSLQAEDVAIYYCQQYYSTLTFGGGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| Full light chain of | MGWSCIILFLVATATGVHSDIVMTQSPDSLAVSLGER | 19 |
| Cilgavimab (with signal | ATINCKSSQSVLYSSNNKNYLAWYQQKPGQPPKLLM | |
| peptide underlined and | YWASTRESGVPDRFSGSGSGAEFTLTISSLQAEDVAI | |
| bolded) | YYCQQYYSTLTFGGGTKVEIKRTVAAPSVFIFPPSDE | |
| | QLKSGTASVVCLLNNFYPREAKVQWKVDNALQSGNSQ | |
| | ESVTEQDSKDSTYSLSSTLTLSKADYEKHKVYACEV | |
| | THQGLSSPVTKSFNRGEC | |

In various aspects, the monoclonal antibodies may comprise a heavy chain comprising an amino acid sequence of SEQ ID NO: 20 (or comprising an amino acid sequence having at least 70%, at least 75%, at least 80%, at least 85%, at least 90%, at least 95%, at least 98%, at least 99% sequence identity to SEQ ID NO: 20). In various aspects, the monoclonal antibody may comprise a heavy chain comprising SEQ ID NO: 22 (or comprising an amino acid sequence having at least 70%, at least 75%, at least 80%, at least 85%, at least 90%, at least 95%, at least 98%, at least 99% sequence identity to SEQ ID NO: 22). SEQ ID NOs 20 and 22 do not comprise the optional signal peptide described above. In some aspects, the signal peptide may be included and, in these aspects, the monoclonal antibody may comprise a light chain comprising or consist of an amino acid sequence of SEQ ID NO: 21 or SEQ ID NO: 23 (or an amino acid sequence having at least 70%, at least 75%, at least 80%, at least 85%, at least 90%, at least 95%, at least 98%, at least 99% sequence identity to SEQ ID NO: 21 or 23. For ease of references, SEQ ID NOs: 20-23 are provided in Table 4 below.

TABLE 4

| | | SEQ |
|---|---|---|
| Description | Sequence | ID NO: |
| Full heavy chain of | QMQLVQSGPEVKKPGTSVKVSCKASGFTFMSSA | 20 |
| tixagevimab (no signal | VQWVRQARGQRLEWIGWIVIGSGNTNYAQKFQE | |
| peptide) | RVTITRDMSTSTAYMELSSLRSEDTAVYYCAAPY | |
| | CSSISCNDGFDIWGQGTMVTVSSASTKGPSVFPL | |
| | APSSKSTSGGTAALGCLVKDYFPEPVTVSWNSG | |
| | ALTSGVHTFPAVLQSSGLYSLSSVTVPSSSLGT | |
| | QTYICNVNHKPSNTKVDKKVEPKSCDKTHTCPPC | |
| | PAPELLGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALP | |
| | APIEKTISKAKGQPREPQVYTLPPSRDELTKNQVS | |
| | LTCLVKGFYPSDIAVEWESNGQPENNYKTTPPVL | |
| | DSDGSFFLYSKLTVDKSRWQQGNVFSCSVMHEA | |
| | LHNHYTQKSLSLSPGK | |
| Full heavy chain of | MGWSCIILFLVATATGVHSQMQLVQSGPEVKKP | 21 |
| tixagevimab (with signal | GTSVKVSCKASGFTFMSSAVQWVRQARGQRLE | |
| peptide) | WIGWIVIGSGNTNYAQKFQERVTITRDMSTSTAY | |
| | MELSSLRSEDTAVYYCAAPYCSSISCNDGFDIWG | |
| | QGTMVTVSSASTKGPSVFPLAPSSKSTSGGTAAL | |
| | GCLVKDYFPEPVTVSWNSGALTSGVHTFPAVLQ | |
| | SSGLYSLSSVVTVPSSSLGTQTYICNVNHKPSNT | |
| | KVDKKVEPKSCDKTHTCPPCPAPELLGGPSVFLF | |
| | PPKPKDTLMISRTPEVTCVVVDVSHEDPEVKFNW | |
| | YVDGVEVHNAKTKPREEQYNSTYRVVSVLTVLH | |
| | QDWLNGKEYKCKVSNKALPAPIEKTISKAKGQPR | |
| | EPQVYTLPPSRDELTKNQVSLTCLVKGFYPSDIA | |
| | VEWESNGQPENNYKTTPPVLDSDGSFFLYSKLT | |
| | VDKSRWQQGNVFSCSVMHEALHNHYTQKSLSL | |
| | SPGK | |
| Full heavy chain of | EVQLVESGGGLVKPGGSLRLSCAASGFTFRDVW | 22 |
| Cilgavimab (no signal | MSWVRQAPGKGLEWVGRIKSKIDGGTTDYAAPV | |
| peptide) | KGRFTISRDDSKNTLYLQMNSLKTEDTAVYYCTT | |
| | AGSYYYDTVGPGLPEGKFDYWGQGTLVTVSSAS | |
| | TKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSCD | |
| | KTHTCPPCPAPELLGGPSVFLFPPKPKDTLMISRT | |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNY | |
| | KTTPPVLDSDGSFFLYSKLTVDKSRWQQGNVFS | |
| | CSVMHEALHNHYTQKSLSLSPGK | |
| Full heavy chain of | MGWSCIILFLVATATGVHSEVQLVESGGGLVKPG | 23 |
| Cilgavimab (with signal | GSLRLSCAASGFTFRDVWMSWVRQAPGKGLEW | |
| peptide) | VGRIKSKIDGGTTDYAAPVKGRFTISRDDSKNTLY | |
| | LQMNSLKTEDTAVYYCTTAGSYYYDTVGPGLPE | |
| | GKFDYWGQGTLVTVSSASTKGPSVFPLAPSSKS | |
| | TSGGTAALGCLVKDYFPEPVTVSWNSGALTSGV | |
| | HTFPAVLQSSGLYSLSSWVTVPSSSLGTQTYICNV | |
| | NHKPSNTKVDKKVEPKSCDKTHTCPPCPAPELL | |
| | GGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTIS | |
| | KAKGQPREPQVYTLPPSRDELTKNQVSLTCLVK | |
| | GFYPSDIAVEWESNGQPENNYKTTPPVLDSDGS | |
| | FFLYSKLTVDKSRWQQGNVFSCSVMHEALHNHY | |
| | TQKSLSLSPGK | |

In any of the above or related aspects, the antibody may have a human-like biantennary GnGn or hybrid MGn glycoform. As to glycoforms, the oligosaccharide structure surrounding the Fc region significantly impacts the therapeutic and anti-inflammatory effects of an mAb. Recombinant Abs are produced in cells engineered for the purpose, typically animal cells. The process of glycosylation in cells is however complex and involves multiple metabolic pathways subject to varying controls. As such, mAbs produced in cells can produce heterogeneous populations of mAbs with different oligosaccharide structures. When recombinant mAbs are produced in cells, the result is often a mixture of proteins with different glycosylation states, or glycoforms. Active glycoforms may account for a relatively small proportion of the resulting mixture. Thus, the inventors' discovery of a method of producing a high proportion of desirable glycoforms and thus functional monoclonal antibodies in plant cells is surprising, unexpected and highly useful.

Further aspects of the present disclosure provide a nucleic acid construct encoding one or more chains (e.g., the heavy and/or light chain) or one or more regions (e.g., the variable or constant regions) of the monoclonal antibodies described herein. Accordingly, a nucleic acid encoding each of the variable regions (e.g., a heavy chain variable region or a light chain variable region) with each optionally further encoding a signal peptide at the N terminus are provided. Likewise, a nucleic acid construct encoding each of the constant regions (e.g., the heavy chain constant regions, IgG1-IgG4, and/or the light chain constant regions) are provided. Various nucleic acid constructs may be envisioned by altering the nucleic acids encoding each of the heavy and light chain components described herein.

For example, in some instances a nucleic acid encoding a heavy chain variable region described above is provided. In some aspects, the nucleic acid has at least 70%, at least 80% or at least 90% sequence identity with SEQ ID NO: 24 or 26. In some aspects, the nucleic acid comprises or consists of SEQ ID NO: 24 or 26. In some aspects, the heavy chain variable region may further comprise a signal peptide and the nucleic acid may further comprise a nucleotide sequence encoding the signal peptide. Accordingly, the nucleic acid encoding a heavy chain variable region (with the signal peptide) may has at least 70%, at least 80% or at least 90% sequence identity with SEQ ID NO: 25 or 27. In some aspects, the nucleic acid encoding a heavy chain variable region (with the signal peptide) may comprise or consist of SEQ ID NO: 25 or 27.

In various aspects, a nucleic acid encoding a heavy chain constant region as described above is provided. For example, in some aspects, the nucleic acid may have at least 70%, at least 80% or at least 90% sequence identity with SEQ ID NO: 28. For instance, the nucleic acid may comprise or consist of SEQ ID NO: 28.

In other aspects, a nucleic acid encoding a light chain variable region described above is provided. In some aspects, the nucleic acid has at least 70%, at least 80% or at least 90% sequence identity with SEQ ID NO: 29 or 31. In some aspects, the nucleic acid comprises or consists of SEQ ID NO: 29 or 31. In some aspects, the light chain variable region may further comprise a signal peptide and the nucleic acid may further comprise a nucleotide sequence encoding the signal peptide. Accordingly, the nucleic acid encoding a light chain variable region (with the signal peptide) may has at least 70%, at least 80% or at least 90% sequence identity with SEQ ID NO: 30 or 32. In some aspects, the nucleic acid encoding a light chain variable region (with the signal peptide) may comprise or consist of SEQ ID NO: 30 or 32.

In still further aspects, a nucleic acid encoding a light chain constant region as described above is provided. In some aspects, the nucleic acid may have at least 70%, at least 80% or at least 90% sequence identity with SEQ ID NO: 33. In some instances, the nucleic acid comprises or consists of SEQ ID NO: 33.

In accordance with various aspects of this disclosure, a nucleic acid construct may be provided wherein the construct comprises a nucleic acid encoding a heavy chain variable region and a nucleic acid encoding a heavy chain constant region, thereby encoding a full-length heavy chain of a monoclonal antibody provided herein. In some aspects, the nucleic acid encoding a full-length heavy chain may have at least 70%, at least 80% or at least 90% sequence identity with SEQ ID NO: 34 or 36. In some aspects, the nucleic acid encoding a full-length heavy chain may comprise or consist of SEQ ID NO: 34 or 36. Accordingly, in some aspects, the nucleic acid construct provided herein may comprise or consist of SEQ ID NO: 34 or 36. In any of these aspects, the full-length heavy chain may further comprise a signal peptide (e.g., at the N-terminus of the heavy chain variable region). A nucleic acid encoding a full-length heavy chain with the signal peptide may, accordingly, have at least 70%, at least 80% or at least 90% sequence identity with SEQ ID NO: 35 or 37. In some aspects, the nucleic acid encoding a full-length heavy chain with the signal peptide may comprise or consist of SEQ ID NO: 35 or 37.

In accordance with various aspects of this disclosure, a nucleic acid construct may be provided wherein the construct comprises a nucleic acid encoding a light chain variable region and a nucleic acid encoding a light chain constant region, thereby encoding a full-length light chain of a monoclonal antibody provided herein. In some aspects, the nucleic acid encoding a full-length light chain may have at least 70%, at least 80% or at least 90% sequence identity with SEQ ID NO: 38 or 40. In some aspects, the nucleic acid encoding a full-length light chain may comprise or consist of SEQ ID NO: 38 or 40. Accordingly, in some aspects, the nucleic acid construct provided herein may comprise or consist of SEQ ID NO: 38 or 40. In any of these aspects, the full-length light chain may further comprise a signal peptide (e.g., at the N-terminus of the light chain variable region). A nucleic acid encoding a full-length light chain with the signal peptide may, accordingly, have at least 70%, at least 80% or at least 90% sequence identity with SEQ ID NO: 39 or 41. In some aspects, the nucleic acid encoding a full-length light chain with the signal peptide may comprise or consist of SEQ ID NO: 39 or 41.

In accordance with these aspects, a nucleic acid construct may be provided encoding a full-length heavy chain and a full-length light chain, optionally with signal peptides at the N-terminus of the heavy and/or light chain. Accordingly, the nucleic acid construct may comprise a nucleic acid encoding a heavy chain selected from SEQ ID NO: 34 (without signal peptide) or SEQ ID NO: 35(with signal peptide) and a nucleic acid encoding a light chain selected from SEQ ID NO: 38 (without signal peptide) or SEQ ID NO: 39 (with signal peptide). In other aspects, the nucleic acid construct may comprise a nucleic acid encoding a heavy chain selected from SEQ ID NO: 36 (without signal peptide) or SEQ ID NO: 37 (with signal peptide) and a nucleic acid encoding a light chain selected from SEQ ID NO:40 (without signal peptide) or SEQ ID NO: 41 (with signal peptide). Other constructs are envisioned having different combinations of the heavy and light chain variable and constant regions described herein.

For ease of reference, nucleic acids encoding one or more portions of the monoclonal antibodies herein (e.g., SEQ ID NOs: 24-41) are provided in Table 5 below.

TABLE 5

| | | SEQ |
|---|---|---|
| Nucleic Acid | | ID |
| Name | Sequence | NO: |
| Tixagevimab heavy | CAAATGCAGTTGGTTCAATCAGGTCCAGAAGTTAAGAAG | 24 |
| chain variable | CCAGGTACATCAGTTAAAGTTTCATGTAAGGCATCAGGAT | |
| region (no signal | TCACTTTCATGTCTTCAGCTGTTCAGTGGGTTAGACAAGC | |
| peptide) | AAGGGGACAAAGACTTGAATGGATTGGTTGGATTGTGAT | |
| | AGGTTCTGGAAATACAAACTATGCACAAAAGTTTCAAGAA | |
| | AGAGTGACTATTACTAGAGATATGTCAACTTCTACTGCTT | |
| | ATATGGAACTTTCTTCTTTGAGATCAGAGGACACAGCTGT | |
| | TTATTATTGCGCAGCACCTTATTGCTCATCTATATCTTGTA | |
| | ATGATGGTTTTGACATTTGGGGACAAGGAACAATGGTTAC | |
| | TGTTTCTTCT | |
| Tixagevimab heavy | ACAATGGGATGGTCTTGTATCATCCTTTTCTTGGTTGCA | 25 |
| chain variable | ACAGCTACTGGTGTTCATTCTCAAATGCAGTTGGTTCAAT | |
| region (with signal | CAGGTCCAGAAGTTAAGAAGCCAGGTACATCAGTTAAAG | |
| peptide) | TTTCATGTAAGGCATCAGGATTCACTTTCATGTCTTCAGC | |
| | TGTTCAGTGGGTTAGACAAGCAAGGGGACAAAGACTTGA | |
| | ATGGATTGGTTGGATTGTGATAGGTTCTGGAAATACAAAC | |
| | TATGCACAAAAGTTTCAAGAAAGAGTGACTATTACTAGAG | |
| | ATATGTCAACTTCTACTGCTTATATGGAACTTTCTTCTTTG | |
| | AGATCAGAGGACACAGCTGTTTATTATTGCGCAGCACCTT | |
| | ATTGCTCATCTATATCTTGTAATGATGGTTTTGACATTTGG | |
| | GGACAAGGAACAATGGTTACTGTTTCTTCT | |
| Cilgavimab heavy | GAAGTTCAGCTTGTGGAGTCAGGTGGAGGTCTTGTTAAG | 26 |
| chain variable | CCAGGTGGATCACTTAGACTTTCTTGCGCTGCTTCAGGTT | |
| region (no signal | TCACATTCAGGGATGTTTGGATGTCATGGGTGAGACAGG | |
| peptide) | CTCCTGGTAAAGGATTGGAGTGGGTTGGTAGAATAAAAT | |
| | CTAAAATCGATGGAGGTACTACTGATTACGCTGCACCAG | |
| | TGAAGGGAAGGTTTACAATATCAAGAGATGATTCAAAGAA | |
| | TACACTTTACCTTCAAATGAACTCATTGAAAACAGAAGAT | |
| | ACTGCTGTGTACTACTGCACAACTGCTGGATCTTATTATT | |
| | ACGACACAGTTGGACCAGGACTTCCAGAGGGAAAGTTTG | |
| | ACTATTGGGGACAGGGAACTCTTGTTACAGTGTCTTC | |
| Cilgavimab heavy | ACAATGGGATGGTCTTGTATCATCCTTTTCTTGGTTGCAA | 27 |
| chain variable | CAGCTACTGGTGTTCATTCTGAAGTTCAGCTTGTGGAGT | |
| region (with signal | CAGGTGGAGGTCTTGTTAAGCCAGGTGGATCACTTAGAC | |
| peptide) | TTTCTTGCGCTGCTTCAGGTTTCACATTCAGGGATGTTTG | |
| | GATGTCATGGGTGAGACAGGCTCCTGGTAAAGGATTGGA | |
| | GTGGGTTGGTAGAATAAAATCTAAAATCGATGGAGGTACT | |
| | ACTGATTACGCTGCACCAGTGAAGGGAAGGTTTACAATA | |
| | TCAAGAGATGATTCAAAGAATACACTTTACCTTCAAATGA | |
| | ACTCATTGAAAACAGAAGATACTGCTGTGTACTACTGCAC | |
| | AACTGCTGGATCTTATTATTACGACACAGTTGGACCAGGA | |
| | CTTCCAGAGGGAAAGTTTGACTATTGGGGACAGGGAACT | |
| | CTTGTTACAGTGTCTTC | |
| Heavy Chain | GCTAGCACCAAGGGACCTTCTGTTTTTCCACTTGCTCCTT | 28 |
| Constant Region | CTTCTAAGTCTACTTCTGGTGGAACTGCTGCTTTGGGTTG | |
| (IgG1) | TTTGGTGAAAGATTACTTTCCTGAGCCAGTGACCGTTTCT | |
| | TGGAACTCAGGTGCTCTTACATCTGGTGTTCATACTTTCC | |
| | CAGCTGTTCTTCAATCTTCAGGACTTTACTCACTTTCTTCT | |
| | GTTGTTACCGTTCCTTCTTCAAGCTTGGGCACTCAGACCT | |
| | ACATCTGCAATGTGAATCACAAACCCAGCAACACCAAGG | |
| | TTGACAAGAAAGTTGAGCCCAAGTCTTGTGACAAGACTC | |
| | ATACGTGTCCACCGTGCCCAGCACCTGAACTTCTTGGAG | |
| | GACCGTCAGTCTTCTTGTTTCCTCCAAAGCCTAAGGATAC | |
| | CTTGATGATCTCCAGGACTCCTGAAGTCACATGTGTAGTT | |
| | GTGGATGTGAGCCATGAAGATCCTGAGGTGAAGTTCAAC | |
| | TGGTATGTGGATGGTGTGGAAGTGCACAATGCCAAGACA | |
| | AAGCCGAGAGAGGAACAGTACAACAGCACGTACAGGGTT | |
| | GTCTCAGTTCTCACTGTTCTCCATCAAGATTGGTTGAATG | |
| | GCAAAGAGTACAAGTGCAAGGTGTCCAACAAAGCCCTCC | |
| | CAGCCCCCATTGAGAAGACCATTTCCAAAGCGAAAGGGC | |
| | AACCCCGTGAACCACAAGTGTACACACTTCCTCCATCTC | |
| | GCGATGAACTGACCAAGAACCAGGTCAGCTTGACTTGCC | |
| | TGGTGAAAGGCTTCTATCCCTCTGACATAGCTGTAGAGT | |
| | GGGAGAGCAATGGGCAACCGGAGAACAACTACAAGACT | |
| | ACACCTCCCGTTCTCGATTCTGACGGCTCCTTCTTCCTCT | |
| | ACAGCAAGCTCACAGTGGACAAGAGCAGGTGGCAACAA | |
| | GGGAATGTCTTCTCATGCTCCGTGATGCATGAGGCTCTT | |
| | CACAATCACTACACACAGAAGAGTCTCTCCTTGTCTCCG | |
| | GGTAAATGA | |
| Tixagevimab light | GAAATAGTTCTTACTCAATCACCTGGTACTCTTTCTCTTTC | 29 |
| chain variable | ACCTGGTGAGAGAGCTACTCTTTCATGTAGAGCATCTCA | |
| region (no signal | GTCTGTTTCTTCTTCATACTTGGCATGGTATCAGCAAAAA | |
| peptide) | CCAGGACAAGCTCCTAGATTGTTGATATATGGAGCTTCTT | |
| | CAAGAGCTACTGGTATTCCAGATAGGTTCTCAGGTTCTG | |
| | GTTCAGGTACTGATTTCACTCTTACTATCTCAAGACTTGA | |
| | GCCTGAAGATTTTGCAGTGTATTACTGCCAGCATTACGGA | |
| | TCTTCAAGGGGTTGGACTTTTGGACAAGGAACTAAAGTT | |
| | GAGATTAAA | |
| Tixagevimab light | ACAATGGGATGGTCTTGTATCATCCTTTTCTTGGTTGCAA | 30 |
| chain variable | CAGCTACTGGTGTTCATTCTGAAATAGTTCTTACTCAATC | |
| region (with signal | ACCTGGTACTCTTTCTCTTTCACCTGGTGAGAGAGCTACT | |
| peptide) | CTTTCATGTAGAGCATCTCAGTCTGTTTCTTCTTCATACTT | |
| | GGCATGGTATCAGCAAAAACCAGGACAAGCTCCTAGATT | |
| | GTTGATATATGGAGCTTCTTCAAGAGCTACTGGTATTCCA | |
| | GATAGGTTCTCAGGTTCTGGTTCAGGTACTGATTTCACTC | |
| | TTACTATCTCAAGACTTGAGCCTGAAGATTTTGCAGTGTA | |
| | TTACTGCCAGCATTACGGATCTTCAAGGGGTTGGACTTTT | |
| | GGACAAGGAACTAAAGTTGAGATTAAA | |
| Cilgavimab light | ACAATGGGATGGTCTTGTATCATCCTTTTCTTGGTTGCAA | 31 |
| chain variable | CAGCTACTGGTGTTCATTCTGATATCGTGATGACTCAGTC | |
| region (no signal | TCCAGATTCTCTTGCAGTTTCACTTGGAGAGAGAGCAACT | |
| peptide) | ATTAATTGCAAGTCATCACAATCAGTGTTGTATTCTTCTAA | |
| | TAACAAGAACTACCTTGCTTGGTATCAACAAAAGCCAGGA | |
| | CAGCCTCCAAAGTTGTTGATGTACTGGGCATCAACAAGA | |
| | GAGTCAGGTGTTCCAGATAGATTCTCTGGTTCTGGTTCA | |
| | GGAGCTGAGTTTACACTTACAATCTCTTCTCTTCAGGCTG | |
| | AAGATGTTGCAATATATTACTGTCAACAATATTATTCTACT | |
| | TTGACTTTTGGTGGTGGAACTAAAGTTGAGATCAAG | |
| Cilgavimab light | ACAATGGGATGGTCTTGTATCATCCTTTTCTTGGTTGCAA | 32 |
| chain variable | CAGCTACTGGTGTTCATTCTGATATCGTGATGACTCAGTC | |
| region (with signal | TCCAGATTCTCTTGCAGTTTCACTTGGAGAGAGAGCAACT | |
| peptide) | ATTAATTGCAAGTCATCACAATCAGTGTTGTATTCTTCTAA | |
| | TAACAAGAACTACCTTGCTTGGTATCAACAAAAGCCAGGA | |
| | CAGCCTCCAAAGTTGTTGATGTACTGGGCATCAACAAGA | |
| | GAGTCAGGTGTTCCAGATAGATTCTCTGGTTCTGGTTCA | |
| | GGAGCTGAGTTTACACTTACAATCTCTTCTCTTCAGGCTG | |
| | AAGATGTTGCAATATATTACTGTCAACAATATTATTCTACT | |
| | TTGACTTTTGGTGGTGGAACTAAAGTTGAGATCAAG | |
| Light Chain | AGGACTGTGGCAGCACCATCTGTGTTCATATTCCCACCA | 33 |
| Constant Region | TCAGATGAACAACTTAAGTCAGGAACAGCTTCAGTTGTTT | |
| (Human Kappa) | GTTTGCTTAACAACTTTTACCCAAGGGAGGCAAAGGTTCA | |
| | ATGGAAGGTGGATAACGCTTTGCAATCAGGAAACTCTCA | |
| | GGAGTCTGTTACAGAACAGGATTCAAAGGATTCTACTTAC | |
| | TCATTGTCTTCAACATTGACTCTTTCTAAGGCTGATTACG | |
| | AGAAGCACAAGGTTTATGCATGTGAAGTGACTCACCAAG | |
| | GTCTTTCTTCACCAGTTACTAAGTCATTCAATAGAGGAGA | |
| | ATGCTGA | |
| Tixagevimab full | CAAATGCAGTTGGTTCAATCAGGTCCAGAAGTTAAGAAG | 34 |
| heavy chain (no | CCAGGTACATCAGTTAAAGTTTCATGTAAGGCATCAGGAT | |
| signal peptide) | TCACTTTCATGTCTTCAGCTGTTCAGTGGGTTAGACAAGC | |
| | AAGGGGACAAAGACTTGAATGGATTGGTTGGATTGTGAT | |
| | AGGTTCTGGAAATACAAACTATGCACAAAAGTTTCAAGAA | |
| | AGAGTGACTATTACTAGAGATATGTCAACTTCTACTGCTT | |
| | ATATGGAACTTTCTTCTTTGAGATCAGAGGACACAGCTGT | |
| | TTATTATTGCGCAGCACCTTATTGCTCATCTATATCTTGTA | |
| | ATGATGGTTTTGACATTTGGGGACAAGGAACAATGGTTAC | |
| | TGTTTCTTCTGCTAGCACCAAGGGACCTTCTGTTTTTCCA | |
| | CTTGCTCCTTCTTCTAAGTCTACTTCTGGTGGAACTGCTG | |
| | CTTTGGGTTGTTTGGTGAAAGATTACTTTCCTGAGCCAGT | |
| | GACCGTTTCTTGGAACTCAGGTGCTCTTACATCTGGTGTT | |
| | CATACTTTCCCAGCTGTTCTTCAATCTTCAGGACTTTACT | |
| | CACTTTCTTCTGTTGTTACCGTTCCTTCTTCAAGCTTGGG | |
| | CACTCAGACCTACATCTGCAATGTGAATCACAAACCCAG | |
| | CAACACCAAGGTTGACAAGAAAGTTGAGCCCAAGTCTTG | |
| | TGACAAGACTCATACGTGTCCACCGTGCCCAGCACCTGA | |
| | ACTTCTTGGAGGACCGTCAGTCTTCTTGTTTCCTCCAAAG | |
| | CCTAAGGATACCTTGATGATCTCCAGGACTCCTGAAGTC | |
| | ACATGTGTAGTTGTGGATGTGAGCCATGAAGATCCTGAG | |
| | GTGAAGTTCAACTGGTATGTGGATGGTGTGGAAGTGCAC | |
| | AATGCCAAGACAAAGCCGAGAGAGGAACAGTACAACAGC | |
| | ACGTACAGGGTTGTCTCAGTTCTCACTGTTCTCCATCAAG | |
| | ATTGGTTGAATGGCAAAGAGTACAAGTGCAAGGTGTCCA | |
| | ACAAAGCCCTCCCAGCCCCCATTGAGAAGACCATTTCCA | |
| | AAGCGAAAGGGCAACCCCGTGAACCACAAGTGTACACAC | |
| | TTCCTCCATCTCGCGATGAACTGACCAAGAACCAGGTCA | |
| | GCTTGACTTGCCTGGTGAAAGGCTTCTATCCCTCTGACAT | |
| | AGCTGTAGAGTGGGAGAGCAATGGGCAACCGGAGAACA | |
| | ACTACAAGACTACACCTCCCGTTCTCGATTCTGACGGCT | |
| | CCTTCTTCCTCTACAGCAAGCTCACAGTGGACAAGAGCA | |
| | GGTGGCAACAAGGGAATGTCTTCTCATGCTCCGTGATGC | |
| | ATGAGGCTCTTCACAATCACTACACACAGAAGAGTCTCTC | |
| | CTTGTCTCCGGGTAAATGA | |
| Tixagevimab full | ACAATGGGATGGTCTTGTATCATCCTTTTCTTGGTTGCAA | 35 |
| heavy chain (with | CAGCTACTGGTGTTCATTCTCAAATGCAGTTGGTTCAATC | |
| signal peptide) | AGGTCCAGAAGTTAAGAAGCCAGGTACATCAGTTAAAGT | |
| | TTCATGTAAGGCATCAGGATTCACTTTCATGTCTTCAGCT | |
| | GTTCAGTGGGTTAGACAAGCAAGGGGACAAAGACTTGAA | |
| | TGGATTGGTTGGATTGTGATAGGTTCTGGAAATACAAACT | |
| | ATGCACAAAAGTTTCAAGAAAGAGTGACTATTACTAGAGA | |
| | TATGTCAACTTCTACTGCTTATATGGAACTTTCTTCTTTGA | |
| | GATCAGAGGACACAGCTGTTTATTATTGCGCAGCACCTTA | |
| | TTGCTCATCTATATCTTGTAATGATGGTTTTGACATTTGGG | |
| | GACAAGGAACAATGGTTACTGTTTCTTCTGCTAGCACCAA | |
| | GGGACCTTCTGTTTTTCCACTTGCTCCTTCTTCTAAGTCT | |
| | ACTTCTGGTGGAACTGCTGCTTTGGGTTGTTTGGTGAAA | |
| | GATTACTTTCCTGAGCCAGTGACCGTTTCTTGGAACTCAG | |
| | GTGCTCTTACATCTGGTGTTCATACTTTCCCAGCTGTTCT | |
| | TCAATCTTCAGGACTTTACTCACTTTCTTCTGTTGTTACCG | |
| | TTCCTTCTTCAAGCTTGGGCACTCAGACCTACATCTGCAA | |
| | TGTGAATCACAAACCCAGCAACACCAAGGTTGACAAGAA | |
| | AGTTGAGCCCAAGTCTTGTGACAAGACTCATACGTGTCC | |
| | ACCGTGCCCAGCACCTGAACTTCTTGGAGGACCGTCAGT | |
| | CTTCTTGTTTCCTCCAAAGCCTAAGGATACCTTGATGATC | |
| | TCCAGGACTCCTGAAGTCACATGTGTAGTTGTGGATGTG | |
| | AGCCATGAAGATCCTGAGGTGAAGTTCAACTGGTATGTG | |
| | GATGGTGTGGAAGTGCACAATGCCAAGACAAAGCCGAGA | |
| | GAGGAACAGTACAACAGCACGTACAGGGTTGTCTCAGTT | |
| | CTCACTGTTCTCCATCAAGATTGGTTGAATGGCAAAGAGT | |
| | ACAAGTGCAAGGTGTCCAACAAAGCCCTCCCAGCCCCCA | |
| | TTGAGAAGACCATTTCCAAAGCGAAAGGGCAACCCCGTG | |
| | AACCACAAGTGTACACACTTCCTCCATCTCGCGATGAACT | |
| | GACCAAGAACCAGGTCAGCTTGACTTGCCTGGTGAAAGG | |
| | CTTCTATCCCTCTGACATAGCTGTAGAGTGGGAGAGCAA | |
| | TGGGCAACCGGAGAACAACTACAAGACTACACCTCCCGT | |
| | TCTCGATTCTGACGGCTCCTTCTTCCTCTACAGCAAGCTC | |
| | ACAGTGGACAAGAGCAGGTGGCAACAAGGGAATGTCTTC | |
| | TCATGCTCCGTGATGCATGAGGCTCTTCACAATCACTACA | |
| | CACAGAAGAGTCTCTCCTTGTCTCCGGGTAAATGA | |
| Cilgavimab full | GAAGTTCAGCTTGTGGAGTCAGGTGGAGGTCTTGTTAAG | 36 |
| heavy chain (no | CCAGGTGGATCACTTAGACTTTCTTGCGCTGCTTCAGGTT | |
| signal peptide) | TCACATTCAGGGATGTTTGGATGTCATGGGTGAGACAGG | |
| | CTCCTGGTAAAGGATTGGAGTGGGTTGGTAGAATAAAAT | |
| | CTAAAATCGATGGAGGTACTACTGATTACGCTGCACCAG | |
| | TGAAGGGAAGGTTTACAATATCAAGAGATGATTCAAAGAA | |
| | TACACTTTACCTTCAAATGAACTCATTGAAAACAGAAGAT | |
| | ACTGCTGTGTACTACTGCACAACTGCTGGATCTTATTATT | |
| | ACGACACAGTTGGACCAGGACTTCCAGAGGGAAAGTTTG | |
| | ACTATTGGGGACAGGGAACTCTTGTTACAGTGTCTTCTG | |
| | CTAGCACCAAGGGACCTTCTGTTTTTCCACTTGCTCCTTC | |
| | TTCTAAGTCTACTTCTGGTGGAACTGCTGCTTTGGGTTGT | |
| | TTGGTGAAAGATTACTTTCCTGAGCCAGTGACCGTTTCTT | |
| | GGAACTCAGGTGCTCTTACATCTGGTGTTCATACTTTCCC | |
| | AGCTGTTCTTCAATCTTCAGGACTTTACTCACTTTCTTCTG | |
| | TTGTTACCGTTCCTTCTTCAAGCTTGGGCACTCAGACCTA | |
| | CATCTGCAATGTGAATCACAAACCCAGCAACACCAAGGT | |
| | TGACAAGAAAGTTGAGCCCAAGTCTTGTGACAAGACTCA | |
| | TACGTGTCCACCGTGCCCAGCACCTGAACTTCTTGGAGG | |
| | ACCGTCAGTCTTCTTGTTTCCTCCAAAGCCTAAGGATACC | |
| | TTGATGATCTCCAGGACTCCTGAAGTCACATGTGTAGTTG | |
| | TGGATGTGAGCCATGAAGATCCTGAGGTGAAGTTCAACT | |
| | GGTATGTGGATGGTGTGGAAGTGCACAATGCCAAGACAA | |
| | AGCCGAGAGAGGAACAGTACAACAGCACGTACAGGGTT | |
| | GTCTCAGTTCTCACTGTTCTCCATCAAGATTGGTTGAATG | |
| | GCAAAGAGTACAAGTGCAAGGTGTCCAACAAAGCCCTCC | |
| | CAGCCCCCATTGAGAAGACCATTTCCAAAGCGAAAGGGC | |
| | AACCCCGTGAACCACAAGTGTACACACTTCCTCCATCTC | |
| | GCGATGAACTGACCAAGAACCAGGTCAGCTTGACTTGCC | |
| | TGGTGAAAGGCTTCTATCCCTCTGACATAGCTGTAGAGT | |
| | GGGAGAGCAATGGGCAACCGGAGAACAACTACAAGACT | |
| | ACACCTCCCGTTCTCGATTCTGACGGCTCCTTCTTCCTCT | |
| | ACAGCAAGCTCACAGTGGACAAGAGCAGGTGGCAACAA | |
| | GGGAATGTCTTCTCATGCTCCGTGATGCATGAGGCTCTT | |
| | CACAATCACTACACACAGAAGAGTCTCTCCTTGTCTCCG | |
| | GGTAAATGA | |
| Cilgavimab full | ACAATGGGATGGTCTTGTATCATCCTTTTCTTGGTTGCAA | 37 |
| heavy chain (with | CAGCTACTGGTGTTCATTCTGAAGTTCAGCTTGTGGAGT | |
| signal peptide) | CAGGTGGAGGTCTTGTTAAGCCAGGTGGATCACTTAGAC | |
| | TTTCTTGCGCTGCTTCAGGTTTCACATTCAGGGATGTTTG | |
| | GATGTCATGGGTGAGACAGGCTCCTGGTAAAGGATTGGA | |
| | GTGGGTTGGTAGAATAAAATCTAAAATCGATGGAGGTACT | |
| | ACTGATTACGCTGCACCAGTGAAGGGAAGGTTTACAATA | |
| | TCAAGAGATGATTCAAAGAATACACTTTACCTTCAAATGA | |
| | ACTCATTGAAAACAGAAGATACTGCTGTGTACTACTGCAC | |
| | AACTGCTGGATCTTATTATTACGACACAGTTGGACCAGGA | |
| | CTTCCAGAGGGAAAGTTTGACTATTGGGGACAGGGAACT | |
| | CTTGTTACAGTGTCTTCTGCTAGCACCAAGGGACCTTCTG | |
| | TTTTTCCACTTGCTCCTTCTTCTAAGTCTACTTCTGGTGGA | |
| | ACTGCTGCTTTGGGTTGTTTGGTGAAAGATTACTTTCCTG | |
| | AGCCAGTGACCGTTTCTTGGAACTCAGGTGCTCTTACAT | |
| | CTGGTGTTCATACTTTCCCAGCTGTTCTTCAATCTTCAGG | |
| | ACTTTACTCACTTTCTTCTGTTGTTACCGTTCCTTCTTCAA | |
| | GCTTGGGCACTCAGACCTACATCTGCAATGTGAATCACA | |
| | AACCCAGCAACACCAAGGTTGACAAGAAAGTTGAGCCCA | |
| | AGTCTTGTGACAAGACTCATACGTGTCCACCGTGCCCAG | |
| | CACCTGAACTTCTTGGAGGACCGTCAGTCTTCTTGTTTCC | |
| | TCCAAAGCCTAAGGATACCTTGATGATCTCCAGGACTCCT | |
| | GAAGTCACATGTGTAGTTGTGGATGTGAGCCATGAAGAT | |
| | CCTGAGGTGAAGTTCAACTGGTATGTGGATGGTGTGGAA | |
| | GTGCACAATGCCAAGACAAAGCCGAGAGAGGAACAGTAC | |
| | AACAGCACGTACAGGGTTGTCTCAGTTCTCACTGTTCTCC | |
| | ATCAAGATTGGTTGAATGGCAAAGAGTACAAGTGCAAGG | |
| | TGTCCAACAAAGCCCTCCCAGCCCCCATTGAGAAGACCA | |
| | TTTCCAAAGCGAAAGGGCAACCCCGTGAACCACAAGTGT | |
| | ACACACTTCCTCCATCTCGCGATGAACTGACCAAGAACC | |
| | AGGTCAGCTTGACTTGCCTGGTGAAAGGCTTCTATCCCT | |
| | CTGACATAGCTGTAGAGTGGGAGAGCAATGGGCAACCG | |
| | GAGAACAACTACAAGACTACACCTCCCGTTCTCGATTCTG | |
| | ACGGCTCCTTCTTCCTCTACAGCAAGCTCACAGTGGACA | |
| | AGAGCAGGTGGCAACAAGGGAATGTCTTCTCATGCTCCG | |
| | TGATGCATGAGGCTCTTCACAATCACTACACACAGAAGA | |
| | GTCTCTCCTTGTCTCCGGGTAAATGA | |
| Tixagevimab full | GAAATAGTTCTTACTCAATCACCTGGTACTCTTTCTCTTTC | 38 |
| light chain (no | ACCTGGTGAGAGAGCTACTCTTTCATGTAGAGCATCTCA | |
| signal peptide) | GTCTGTTTCTTCTTCATACTTGGCATGGTATCAGCAAAAA | |
| | CCAGGACAAGCTCCTAGATTGTTGATATATGGAGCTTCTT | |
| | CAAGAGCTACTGGTATTCCAGATAGGTTCTCAGGTTCTG | |
| | GTTCAGGTACTGATTTCACTCTTACTATCTCAAGACTTGA | |
| | GCCTGAAGATTTTGCAGTGTATTACTGCCAGCATTACGGA | |
| | TCTTCAAGGGGTTGGACTTTTGGACAAGGAACTAAAGTT | |
| | GAGATTAAAAGGACTGTGGCAGCACCATCTGTGTTCATAT | |
| | TCCCACCATCAGATGAACAACTTAAGTCAGGAACAGCTTC | |
| | AGTTGTTTGTTTGCTTAACAACTTTTACCCAAGGGAGGCA | |
| | AAGGTTCAATGGAAGGTGGATAACGCTTTGCAATCAGGA | |
| | AACTCTCAGGAGTCTGTTACAGAACAGGATTCAAAGGATT | |
| | CTACTTACTCATTGTCTTCAACATTGACTCTTTCTAAGGCT | |
| | GATTACGAGAAGCACAAGGTTTATGCATGTGAAGTGACT | |
| | CACCAAGGTCTTTCTTCACCAGTTACTAAGTCATTCAATA | |
| | GAGGAGAATGCTGA | |
| Tixagevimab full | ACAATGGGATGGTCTTGTATCATCCTTTTCTTGGTTGCAA | 39 |
| light chain (with | CAGCTACTGGTGTTCATTCTGAAATAGTTCTTACTCAATC | |
| signal peptide) | ACCTGGTACTCTTTCTCTTTCACCTGGTGAGAGAGCTACT | |
| | CTTTCATGTAGAGCATCTCAGTCTGTTTCTTCTTCATACTT | |
| | GGCATGGTATCAGCAAAAACCAGGACAAGCTCCTAGATT | |
| | GTTGATATATGGAGCTTCTTCAAGAGCTACTGGTATTCCA | |
| | GATAGGTTCTCAGGTTCTGGTTCAGGTACTGATTTCACTC | |
| | TTACTATCTCAAGACTTGAGCCTGAAGATTTTGCAGTGTA | |
| | TTACTGCCAGCATTACGGATCTTCAAGGGGTTGGACTTTT | |
| | GGACAAGGAACTAAAGTTGAGATTAAAAGGACTGTGGCA | |
| | GCACCATCTGTGTTCATATTCCCACCATCAGATGAACAAC | |
| | TTAAGTCAGGAACAGCTTCAGTTGTTTGTTTGCTTAACAA | |
| | CTTTTACCCAAGGGAGGCAAAGGTTCAATGGAAGGTGGA | |
| | TAACGCTTTGCAATCAGGAAACTCTCAGGAGTCTGTTACA | |
| | GAACAGGATTCAAAGGATTCTACTTACTCATTGTCTTCAA | |
| | CATTGACTCTTTCTAAGGCTGATTACGAGAAGCACAAGGT | |
| | TTATGCATGTGAAGTGACTCACCAAGGTCTTTCTTCACCA | |
| | GTTACTAAGTCATTCAATAGAGGAGAATGCTGA | |
| Cilgavimab full light | GATATCGTGATGACTCAGTCTCCAGATTCTCTTGCAGTTT | 40 |
| chain (no signal | CACTTGGAGAGAGAGCAACTATTAATTGCAAGTCATCACA | |
| peptide) | ATCAGTGTTGTATTCTTCTAATAACAAGAACTACCTTGCTT | |
| | GGTATCAACAAAAGCCAGGACAGCCTCCAAAGTTGTTGA | |
| | TGTACTGGGCATCAACAAGAGAGTCAGGTGTTCCAGATA | |
| | GATTCTCTGGTTCTGGTTCAGGAGCTGAGTTTACACTTAC | |
| | AATCTCTTCTCTTCAGGCTGAAGATGTTGCAATATATTACT | |
| | GTCAACAATATTATTCTACTTTGACTTTTGGTGGTGGAAC | |
| | TAAAGTTGAGATCAAGAGGACTGTGGCAGCACCATCTGT | |
| | GTTCATATTCCCACCATCAGATGAACAACTTAAGTCAGGA | |
| | ACAGCTTCAGTTGTTTGTTTGCTTAACAACTTTTACCCAA | |
| | GGGAGGCAAAGGTTCAATGGAAGGTGGATAACGCTTTGC | |
| | AATCAGGAAACTCTCAGGAGTCTGTTACAGAACAGGATT | |
| | CAAAGGATTCTACTTACTCATTGTCTTCAACATTGACTCTT | |
| | TCTAAGGCTGATTACGAGAAGCACAAGGTTTATGCATGT | |
| | GAAGTGACTCACCAAGGTCTTTCTTCACCAGTTACTAAGT | |
| | CATTCAATAGAGGAGAATGCTGA | |
| Cilgavimab full light | ACAATGGGATGGTCTTGTATCATCCTTTTCTTGGTTGCAA | 41 |
| chain (with signal | CAGCTACTGGTGTTCATTCTGATATCGTGATGACTCAGTC | |
| peptide) | TCCAGATTCTCTTGCAGTTTCACTTGGAGAGAGAGCAACT | |
| | ATTAATTGCAAGTCATCACAATCAGTGTTGTATTCTTCTAA | |
| | TAACAAGAACTACCTTGCTTGGTATCAACAAAAGCCAGGA | |
| | CAGCCTCCAAAGTTGTTGATGTACTGGGCATCAACAAGA | |
| | GAGTCAGGTGTTCCAGATAGATTCTCTGGTTCTGGTTCA | |
| | GGAGCTGAGTTTACACTTACAATCTCTTCTCTTCAGGCTG | |
| | AAGATGTTGCAATATATTACTGTCAACAATATTATTCTACT | |
| | TTGACTTTTGGTGGTGGAACTAAAGTTGAGATCAAGAGG | |
| | ACTGTGGCAGCACCATCTGTGTTCATATTCCCACCATCAG | |
| | ATGAACAACTTAAGTCAGGAACAGCTTCAGTTGTTTGTTT | |
| | GCTTAACAACTTTTACCCAAGGGAGGCAAAGGTTCAATG | |
| | GAAGGTGGATAACGCTTTGCAATCAGGAAACTCTCAGGA | |
| | GTCTGTTACAGAACAGGATTCAAAGGATTCTACTTACTCA | |
| | TTGTCTTCAACATTGACTCTTTCTAAGGCTGATTACGAGA | |
| | AGCACAAGGTTTATGCATGTGAAGTGACTCACCAAGGTC | |
| | TTTCTTCACCAGTTACTAAGTCATTCAATAGAGGAGAATG | |
| | CTGA | |

The present disclosure provides antibodies binding to a receptor binding domain (RBD) of a coronavirus spike protein. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion (e.g., chimeric antigen receptor or CAR), humanized antibodies, chimeric antibodies, diabodies, single domain antibody (e.g., a VH only antibody such as a nanobody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody encompassed herein may include an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively.

An antibody molecule encompassed herein may comprise a heavy chain variable region (VH) and a light chain variable region (VL). The VH and VL regions can be further subdivided into regions of hypervariability, also known as “complementarity determining regions” (“CDR”), interspersed with regions that are more conserved, which are known as “framework regions” (“FR”). Each VH and VL may be composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) U.S. Department of Health and Human Services, NIH Publication No. 91-3242; Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004).

In certain embodiments, antibodies described herein may specifically bind to a corresponding target antigen (e.g., a RBD of a coronavirus spike protein) or an epitope thereof. An antibody that “specifically binds” to an antigen or an epitope is a term well understood in the art. A molecule is said to exhibit “specific binding” if it reacts more frequently, more rapidly, with greater duration, and/or with greater affinity with a particular target antigen than it does with alternative targets. An antibody “specifically binds” to a target antigen or epitope if it binds with greater affinity, avidity, more readily, and/or with greater duration than it binds to other substances. As an example, an antibody that specifically (or preferentially) binds to an antigen (e.g., a RBD of a coronavirus spike protein) or an antigenic epitope therein is an antibody that binds this target antigen with greater affinity, avidity, more readily, and/or with greater duration than it binds to other antigens or other epitopes in the same antigen. It is also understood with this definition that, as an example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In certain embodiments, provided herein are antibodies which can bind a receptor binding domain (RBD) of a coronavirus spike protein. In some embodiments, antibodies herein can be isolated antibodies that bind a RBD of a coronavirus spike protein. In some embodiments, isolated antibodies herein may bind a RBD of a coronavirus spike protein, wherein the coronavirus may be one or more human coronaviruses (HCoVs). In some embodiments, isolated antibodies herein may bind a RBD of a coronavirus spike protein, wherein the coronavirus may be human coronavirus-229E (HCoV-229E), human coronavirus-NL63 (HCoV-NL63), human coronavirus-OC43 (HCoV-OC43), human coronavirus-HKU1 (HCoV-HKU1), Middle East respiratory syndrome coronavirus (MERS-CoV), severe acute respiratory syndrome coronavirus (SARS-CoV), and/or severe acute respiratory syndrome coronavirus 2 (SARS-CoV-2). In some embodiments, isolated antibodies herein may bind a RBD of a coronavirus spike protein, wherein the coronavirus is SARS-CoV-2.

In certain embodiments, antibodies described herein may have a suitable binding affinity for a target antigen (e.g., a RBD of a coronavirus spike protein) or an epitope thereof. As used herein, “binding affinity” refers to the apparent association constant or KA. The KA is the reciprocal of the dissociation constant (KD). In some embodiments, an antibody described herein may have a binding affinity (KD) of at least about 100 nM, at least about 10 nM, at least about 1 nM, at least about 0.1 nM, or lower for a RBD of a coronavirus spike protein. In some embodiments, an antibody described herein may have a binding affinity (KD) between about 100 nM to about 0.1 nM (e.g., about 100 nM, about 75 nM, about 50 nM, about 25 nM, about 10 nM, about 5 nM, about 1 nM, about 0.75 nM, about 0.5 nM, about 0.25 nM, about 0.1 nM) fora RBD of a coronavirus spike protein. In some embodiments, an antibody described herein may have a binding affinity (KD) between about 50 nM to about 40 nM (e.g., about 50 nM, about 49 nM, about 48 nM, about 47 nM, about 46 nM, about 45 nM, about 44 nM, about 43 nM, about 42 nM, about 41 nM, about 40 nM) for a RBD of a coronavirus spike protein. In some embodiments, an antibody described herein may have a binding affinity (KD) between about 50 nM to about 40 nM (e.g., about 50 nM, about 49 nM, about 48 nM, about 47 nM, about 46 nM, about 45 nM, about 44 nM, about 43 nM, about 42 nM, about 41 nM, about 40 nM) for a RBD of a SARS-CoV-2 spike protein. In some embodiments, binding affinity (or binding specificity) can be determined by a variety of methods including equilibrium dialysis, equilibrium binding, gel filtration, ELISA, surface plasmon resonance, and/or spectroscopy (e.g., using a fluorescence assay).

In certain embodiments, antibodies described herein may block coronavirus (e.g., SARS-CoV-2) replication. In certain embodiments, antibodies described herein may decrease coronavirus (e.g., SARS-CoV-2) replication compared to coronavirus replication in the absence of an antibody herein. In certain embodiments, antibodies described herein may decrease coronavirus (e.g., SARS-CoV-2) replication by about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to coronavirus replication in the absence of an antibody herein.

In certain embodiments, antibodies described herein may inhibit coronavirus (e.g., SARS-CoV-2) infectivity. In some embodiments, antibodies described herein may have a suitable inhibition of coronavirus (e.g., SARS-CoV-2) infectivity, wherein suitable inhibition may be about 5% to about 99% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%) compared to coronavirus replication in the absence of an antibody herein.

In certain embodiments, antibodies described herein may have neutralizing activity against a coronavirus (e.g., SARS-CoV-2) Spike protein. In some embodiments, antibodies described herein may have neutralizing activity against a coronavirus (e.g., SARS-CoV-2) Spike protein with IC50 less than about 150 ng/mL. In some embodiments, antibodies described herein may have neutralizing activity against a coronavirus (e.g., SARS-CoV-2) Spike protein with IC50 ranging from about 0.1 ng/ml to about 150 ng/mL (e.g., about 0.1 ng/ml, about 0.25 ng/ml, about 0.5 ng/ml, about 1 ng/ml, about 2.5 ng/ml, about 5 ng/ml, about 7.5 ng/ml, about 10 ng/ml, about 25 ng/ml, about 50 ng/ml, about 75 ng/ml, about 100 ng/ml, about 125 ng/ml, about 150 ng/ml).

In certain embodiments, antibodies described herein may bind to the same epitope of a coronavirus (e.g., SARS-CoV-2) Spike protein as any of the exemplary antibodies described herein. An “epitope” refers to the site on a target antigen that is recognized and bound by an antibody (e.g., a RBD of a coronavirus spike protein). In some embodiments, an epitope herein can be entirely composed of amino acid components, entirely composed of chemical modifications of amino acids of the protein (e.g., glycosyl moieties), and/or composed of combinations thereof. In some embodiments, epitopes herein can be overlapping epitopes that include at least one common amino acid residue. In some embodiments, an epitope herein can be linear, which may be about 5 to about 15 (e.g., about 5, about 6, about 7, about 8, about 9, about 10, about 11, about 12, about 13, about 14, about 15) about amino acids in length. In some embodiments, an epitope herein can be conformational. The epitope to which an antibody binds can be determined by routine technology, for example, the epitope mapping method (See, e.g., descriptions below).

In certain embodiments, antibodies described herein may have the same heavy chain CDRs, and/or light chain CDRs as any of the exemplary antibodies described herein. Two antibodies having the same heavy chain CDRs, and/or light chain CDRs means that their CDRs are identical when determined by the same approach (e.g., the Kabat approach, the Chothia approach, the AbM approach, the Contact approach, or the IMGT approach). In certain embodiments, antibodies described herein may have the same heavy chain and/or light chain as any of the exemplary antibodies described herein. In certain embodiments, antibodies disclosed herein may share one or more amino acid sequences provided in Tables 1 to 4 above.

In some embodiments, antibodies herein may comprise heavy chains that are at least about 80% (e.g., about 85%, about 90%, about 95%, about 98%) sequence identity, individually or collectively, as compared with the heavy chain of an exemplary antibody described herein. In some embodiments, antibodies herein may comprise light chains that are at least about 80% (e.g., about 85%, about 90%, about 95%, about 98%) sequence identity, individually or collectively, as compared with the light chains of an exemplary antibody described herein. In some embodiments, antibodies herein may comprise heavy chain CDRs that are at least about 80% (e.g., about 85%, about 90%, about 95%, about 98%) sequence identity, individually or collectively, as compared with the heavy chain CDRs of an exemplary antibody described herein. In some embodiments, antibodies herein may comprise light chain CDRs that are at least about 80% (e.g., about 85%, about 90%, about 95%, about 98%) sequence identity, individually or collectively, as compared with the light chain CDRs of an exemplary antibody described herein.

The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Two strains of SARSr-CoV have caused serious outbreaks of severe respiratory diseases in humans: SARS-CoV, which caused an outbreak of severe acute respiratory syndrome (SARS) between 2002 and 2003, and SARS-CoV-2, which has caused the coronavirus disease 2019 (COVID-19) pandemic. There are hundreds of strains of SARSr-CoV known only to infect non-human species; bats are a major reservoir of many strains of SARS-related coronaviruses.

In some embodiments in accordance with the various aspects of the present disclosure, the SARSr-CoV is a strain of SARSr-CoV that causes coronavirus disease in humans.

In some embodiments, the SARSr-CoV is SARS-CoV or a variant thereof, or SARS-CoV-2 or a variant thereof. In some embodiments, the SARSr-CoV is SARS-CoV-2 or a variant thereof.

In some embodiments herein, “SARS-CoV” refers to the SARSr-CoV strain having the nucleotide sequence with a GenBank Accession No.: AY278488.2 (“SARS coronavirus BJ01, complete genome”) see, e.g., Wu et al., Genomics Proteomics Bioinformatics 1 (2), 131-144 (2003). A variant of SARS-CoV may comprise a nucleotide sequence having at least 80% sequence identity, e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to the nucleotide sequence with a GenBank Accession No.: AY278488.2.

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using the publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such softwares, the default parameters, e.g. for gap penalty and extension penalty, are preferably used.

As used herein, “SARS-CoV-2” refers to the SARSr-CoV strain having the nucleotide sequence with a GenBank Accession No.: MN996527.1 (“Severe acute respiratory syndrome coronavirus 2 isolate WIV02, complete genome”), reported in Zhou et al., Nature (2020) 579: 270-273. A variant of SARS-CoV-2 may comprise a nucleotide sequence having at least 80% sequence identity, e.g. one of at least 81%, 82%, 83%, 84%, 85%, 86%, 8′7%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to the nucleotide sequence with a GenBank Accession No.: MN996527.1.

### I. Methods

(i) Treatments

In one aspect, the terms “treatment” and “treating” refer to abating a coronavirus infection in a human or animal subject. By virtue of the administration of any of the MAbs or compositions described herein, the viral infection rate (infectious titer) in the subject decreases and may be eliminated. The terms “treatment” and “treating” also refers to attenuating symptoms associated with the viral infection (e.g., respiratory syndrome, kidney failure, fever, and other symptoms relating to coronavirus infections).

The terms “effective amount”, “therapeutically effective amount” or “effective dose” or related terms may be used interchangeably and refer to an amount of the therapeutic agent that when administered to a subject, is sufficient to affect a measurable improvement or prevention of a disease or disorder associated with coronavirus infection. For example, administering an effective dose sufficient to inhibit the proliferation and/or replication of the coronavirus, and/or the development of the viral infection within the subject. Therapeutically effective amounts of the therapeutic agents provided herein, when used alone or in combination with an antiviral agent, will vary depending upon the relative activity of the therapeutic agent, and depending upon the subject and disease condition being treated, the weight and age and sex of the subject, the severity of the disease condition in the subject, the manner of administration and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for age as well as the body weight, general health, sex, diet, time of administration, drug interaction, and the severity of the disease may be necessary.

As used herein, a virus variant, such as SARS-CoV-2 variant means an isolate of a virus, such as a SARS-Cov-2 Washington/Wuhan-Hu-1 (also referred to as WA1/2020, used interchangeably throughout), as well as currently known, emerging, and/or yet-to-emerge isolates, each independently having one or more mutations with respect to a reference virus, such as a SARS-CoV-2 Washington/Wuhan-Hu-lisolate, from which it is derived. Thus, a used herein, a SARS-CoV-2 variant may be a Washington/Wuhan-Hu-1 isolate, as well as a variant in reference to a Washington/Wuhan-Hu-1 isolate. A variant, for example, may typically have multiple mutations with respect to a Washington/Wuhan-Hu-1 isolate which, for the purposes herein, comprises and RBD and/or and S protein comprising such RBD, of a coronavirus isolate, such as a Washington/Wuhan-Hu-1 isolate, and/or variants thereof.

A subject in need of treatment may be a human subject or other subject having, suspected of having or at risk of having a viral infection or viral disease/disorder. A human subject can be of any age, such as an infant, a toddler, a pre-schooler, a pre-teen, a teenager, a youth, or an adult. A human subject may have an age from 0 to 2 years old, from 2 to 12 years old, from 12 to 16 years old, from 16 to 18 years old, from 18 to 21 years old, from 21 to 30 years old, from 30 to 50 years old, from 50 to 60 years old, or older than 60 years.

In some instances, any one or more of the monoclonal antibodies or compositions described herein may be administered to the subject over several days to several weeks or longer at a suitable interval, depending on the condition, the treatment is sustained until a desired suppression of symptoms occurs or until sufficient therapeutic levels are achieved to alleviate a target disease or disorder, or a symptom thereof. An exemplary dosing regimen comprises administering one or more initial doses at a suitable interval over a suitable period. If necessary, multiple maintenance doses can be given to the subject at a suitable interval over a suitable period of time. However, other dosage regimens may be useful, depending on the pattern of pharmacokinetic decay that the practitioner wishes to achieve. For example, dosing from one to twenty-four times a day or a week is contemplated. In some aspects, the dose may be administered to the patient 1, 2, 3, 4, or 5 times per day. In particular, the composition may be administered orally.

In some aspects, dosing frequency can be continuously for the period medically or therapeutically needed, every one hour, every two hours, four times a day, three times a day, twice a day, once a day, once every other day, once every week, once every 2 weeks, once every 4 weeks, once every 2 months, once every 3 months or only given once. The dosing regimen can vary over time.

The particular dosage regimen, i.e., dose, timing and repetition, will depend on the particular individual and that individual's medical history, as well as the properties of the individual agents (such as the half-life of the agent, and other considerations well known in the art).

Conventional methods, known to those of ordinary skill in the art of medicine, can be used to administer the composition (e.g., a pharmaceutical composition, a health food composition, a nutraceutical composition or a medical food composition) to the subject, depending upon the type of viral infection disease to be treated or the site of the disease. This composition can also be administered via other conventional routes, e.g., administered orally, parenterally, by inhalation spray, topically, rectally, nasally, buccally, or via an implanted reservoir. The term “parenteral” as used herein includes subcutaneous, intracutaneous, intravenous, intramuscular, intraarticular, intra-arterial, intrasynovial, intrasternal, intrathecal, intralesional, and intracranial injection or infusion techniques.

In one aspect, the method of treatment includes treating an asymptomatic subject, or a symptomatic subject. Symptoms of a subject may include anosmia, loss of taste, headache, fever, sore throat, general malaise, fatigue, joint pain, gastrointestinal disturbance, cough, dyspnoea, respiratory stress, or any combination thereof.

(ii) Combined or Combination Therapy

Also provided herein are combined therapies using any of the compositions described herein and a second therapeutic agent, such as those described herein. The term combination therapy, as used herein, embraces administration of these agents (e.g., the diphenyl compound and an antiviral agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route including, but not limited to, oral routes, intravenous routes, intramuscular, subcutaneous routes, direct absorption through mucous membrane tissues, and pulmonary delivery routes. The agents can be administered by the same route or by different routes. For example, a first agent (e.g., a composition described herein) can be administered by pulmonary delivery routes, and a second agent (e.g., an antiviral agent) can be administered intravenously.

Examples of the additional pharmaceutical agent selected from a viral entry inhibitor, a viral uncoating inhibitor, a viral reverse transcriptase inhibitor, a viral protein synthesis inhibitor, a viral protease inhibitor, a viral polymerase inhibitor, a viral integrase inhibitor, an interferon, or the combination thereof. Examples of viral entry inhibitor include, but are not limited to, maraviroc, enfuvirtide, ibalizumab, fostemsavir, plerixafor, epigallocatechin gallate, vicriviroc, aplaviroc, maraviroc, tromantadine, nitazoxanide, umifenovir, and podofilox. Examples of viral uncoating inhibitor include, but are not limited to, amantadine, rimantadine, and pleconaril. Examples of reverse transcriptase inhibitor include, but are not limited to zidovudine, didanosine, zalcitabine, stavudine, lamivudine, abacavir, emtricitabine, entecavir, truvada, nevirapine, raltegravir, and tenofovir disoproxil. Examples of viral protease inhibitor, include but are not limited to, fosamprenavir, ritonavir, atazanavir, nelfinavir, indinavir, saquinavir, saquinavir, famciclovir, fomivirsen, lopinavir, ribavirin, darunavir, oseltamivir, and tipranavir. Examples of viral polymerase inhibitor, include but are not limited to, amatoxins, rifamycin, cytarabine, fidaxomicin, tagetitoxin, foscarnet sodium, idoxuridine, penciclovir, sofosbuvir, trifluridine, valacyclovir, valganciclovir, vidarabine, and remdesivir. Examples of viral integrase inhibitor, include but are not limited to, raltegarvir, elvitegravir, dolutegravir, bictegravir, and cabotegravir. Examples of interferon, include but are not limited to, type I interferon, type II interferon, type Ill interferon, and peginterferon alfa-2a. Other anti-viral agents, include, but not limited to, REGN10987 (imdevimab), REGN10933 (casirivimab), LY-CoV555 (bamlanivimab), CB6 (etesevimab), Brii-196 (amubarvimab), Brii-198 (romlusevimab), S309 (sotrovimab), LY-CoV1404 (bebtelovimab), ADG-2, DH1047, S2X259, and CAB-A17, ZCB11.

As used herein, the term “sequential” means, unless otherwise specified, characterized by a regular sequence or order, e.g., if a dosage regimen includes the administration of a composition and an antiviral agent, a sequential dosage regimen could include administration of the composition before, simultaneously, substantially simultaneously, or after administration of the antiviral agent, but both agents will be administered in a regular sequence or order. The term “separate” means, unless otherwise specified, to keep apart one from the other. The term “simultaneously” means, unless otherwise specified, happening or done at the same time, i.e., the agents as disclosed herein are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the agents described herein.

Combination therapy can also embrace the administration of the agents described herein (e.g., the composition and an antiviral agent) in further combination with other biologically active ingredients (e.g., a different antiviral agent) and non-drug therapies.

It should be appreciated that any combination of a composition described herein and a second therapeutic agent (e.g., an antiviral agent) may be used in any sequence for treating a target disease. The combinations described herein may be selected on the basis of a number of factors, which include but are not limited to the effectiveness of inhibiting virus or at least one symptom associated with the virus infection, As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

(iii) Methods of Making

In another aspect, the present disclosure provides a method of making a SARS-CoV-2 mAb variant from a parent SARS-CoV-2 mAb produced in a mammalian cell. The method includes (i) fusing a nucleic acid sequence encoding a variable region of the parent SARS-CoV-2 mAb to a nucleic acid sequence encoding a human IgG backbone; (ii) restoring a nucleic acid sequence encoding an FcγR domain of the variable region of the parent SARS-CoV-2 mAb to form an expression construct for expressing the SARS-CoV-2 mAb variant; (iii) introducing the expression construct into a plant; and (iv) maintaining the plant for a time and under conditions sufficient for the plant to express the SARS-CoV-2 mAb variant. In one aspect, the parent SARS-CoV-2 mAb can be tixagevimab or cilgavimab, or both can be expressed in the plant. The nucleic acids can be any of those as described herein. A vector as described herein and in the examples may be used to introduce the nucleic acids into the plant cell, using methods known in the art. In one aspect, the plant is a N. benthamiana plant having reduced expression of xylosyltransferase and/or fucosyltransferase, and optionally is ΔXFT N. benthamiana plant. In another aspect, the plant is a N. benthamiana plant having reduced expression of β 1,2-xylosyltransferase (XylT) and/or α 1,3-fucosyltransferase (FucT).

### II. Pharmaceutical Formulations

One aspect of the disclosure encompasses a pharmaceutical formulation and/or dosage form, which comprises any of the MAbs, nucleic acid constructs, vectors or cells described herein, with a pharmaceutically acceptable carrier, diluent or excipient. A pharmaceutically acceptable carrier, diluent and/or excipient may be selected from but is not limited to, water, NaCl, a normal saline solution, a Ringer's solution, a lactated Ringer's solution, a normal sucrose, a normal glucose, a binder, a filler, a disintegrant, a lubricant, an alcohol, an oil, a gelatin, a carbohydrate, starch, a fatty acid, a fatty acid ester, a hydroxymethycellulose, a polyvinyl pyrrolidine, a lipid, a lipid nanoparticle (LNPs), a polymer, or a peptide. The carrier may comprise a cationic lipid, a non-cationic lipid, a lipid nanoparticle (LNPs), a polyamine, a dendrimer, a polyethylenimine (PEI), a polyamidoamine (PAMAM) dendrimer, a polysaccharide or PLGA mixed with cationic lipids, chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation.

Non-limiting examples of excipients such as binders suitable for the formulations of various aspects include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohols, polyethylene glycol, polyols, saccharides, oligosaccharides, polypeptides, oligopeptides, and combinations thereof. The polypeptide may be any arrangement of amino acids ranging from about 100 to about 300,000 Daltons. The binder can be introduced into the mixture to be granulated in a solid form, including but not limited to a crystal, a particle, a powder, or any other finely divided solid form known in the art. Alternatively, the binder can be dissolved or suspended in a solvent and sprayed onto the mixture in a granulation device as a binder fluid during granulation.

Non-limiting examples of diluents (also referred to as “fillers” or “thinners”) include carbohydrates, inorganic compounds, and biocompatible polymers, such as polyvinylpyrrolidone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

Pharmaceutically acceptable carriers, diluents and/or excipients include disintegrents which can be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

Pharmaceutically acceptable carriers, diluents and/or excipients include preservatives which include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

Pharmaceutically acceptable carriers, diluents and/or excipients include flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot. Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety. Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevie rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Pharmaceutically acceptable carriers, diluents and/or excipients include lubricant compositions which may be utilized to lubricate ingredients that form a pharmaceutical composition. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The pharmaceutical composition will generally comprise from about 0.01% to about 10% by weight of a lubricant. In some aspects, the pharmaceutical composition will comprise from about 0.1% to about 5% by weight of a lubricant. In a further aspect, the pharmaceutical composition will comprise from about 0.5% to about 2% by weight of a lubricant.

Pharmaceutically acceptable carriers, diluents and/or excipients include lubricant dispersants and colorants. Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects of the disclosure.

Pharmaceutically acceptable carriers, diluents and/or excipients include pH modifiers such as citric acid, acetic acid, tartaric acid, malic acid, fumaric acid, lactic acid, phosphoric acid, sorbic acid, benzoic acid, sodium carbonate and sodium bicarbonate.

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na₂EDTA).

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na₂EDTA, and sulfites including but not limited to sulfur dioxide, sodium bisulfite, and potassium hydrogen sulfite.

Release-controlling polymers may be included in the various aspects of the solid dosage pharmaceutical compositions incorporating compounds according to this disclosure. In one aspect, the release-controlling polymers may be used as a tablet coating. In other aspects, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, arabic gum, carrageens gum, ghafti gum, guar gum, karaya gum, locust bean gum, okra gum, tragacanth gum, scleroglucan gum, xanthan gum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

A solid dosage form comprising an MAb according to this disclosure may comprise a coating, wherein such a coating may control release of the compound, act as a moisture barrier, or buffer or modify pH. A “control releasing coat” or “controlled release coat” as used herein is defined to mean a functional coat which can for example comprise at least one pH independent polymer, pH dependent polymer (for example enteric or reverse enteric type polymers), soluble polymer, insoluble polymer, lipids, lipidic materials, or combinations thereof. The coating, when applied onto a dosage form, may slow (for example when applied to a normal release matrix dosage form), further slow (for example when applied to a controlled release matrix dosage form) or modify the rate of release of a compound according to this disclosure when applied to an uncoated dosage form. For example, the control releasing coat can be designed such that when the control releasing coat is applied to a dosage form, the dosage form in conjunction with the control releasing coat can exhibit the release of the compound according to this disclosure, such as a “modified-release”, “controlled-release”, “sustained-release”, “extended-release”, “delayed-release”, “prolonged-release,” or combinations thereof. The “control releasing coat” may optionally comprise additional materials that may alter the functionality of the control releasing coat.

The term “moisture barrier” as used herein is one which impedes or retards the absorption of moisture. Compounds according to this disclosure may be hygroscopic and, as such, may be susceptible to decomposition over time under highly humid conditions. The proportion of the components of the moisture barrier and the amount of the moisture barrier optionally applied onto the control-releasing coating or onto the core are typically such that the moisture barrier does not fall within the USP definition and requirement for an enteric coat. Suitably, the moisture barrier may comprise an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier may additionally comprise other conventional inert excipients, which may improve processing of an extended-release formulation.

Coating and matrix materials which may be used in accordance with the disclosure are those known in the art for use in controlled-release formulations, such as synthetic polymers of the polyvinyl type, e.g., polyvinylchloride, polyvinylacetate and copolymers thereof, polyvinylalcohol, and polyvinylpyrrolidone; synthetic polymers of the polyethylene type, e.g., polyethylene and polystyrene; acrylic acid polymers; biopolymers or modified biopolymers, such as cellulosic polymers, shellac and gelatin; fats, oils, higher fatty acids and higher alcohols (i.e., acids and alcohols containing alkyl chains of at least 10 carbon atoms), for example aluminum monostearate, cetylalcohol, hydrogenated beef tallow, hydrogenated castor oil, 12-hydroxystearl alcohol, glyceryl mono- or dipalmitate; glyceryl mono-, di- or tristearate; myristyl alcohol, stearic acid, stearyl alcohol, and polyethyleneglycols; waxes; sugars and sugar alcohols.

The pH-buffering properties of a coating may be strengthened by introducing into the coating substances chosen from a group of compounds usually used in antacid formulations, for example magnesium oxide, hydroxide or carbonate, aluminum or calcium hydroxide, carbonate or silicate; composite aluminum/magnesium compounds, for example Al₂O₃·6MgO·CO₂·12H₂O, (Mg₆Al₂ (OH)₁₆CO₃·4H₂O), MgO·Al₂O₃·2SiO₂·nH₂O, aluminum bicarbonate coprecipitate or similar compounds; or other pharmaceutically acceptable pH-buffering compounds, for example the sodium, potassium, calcium, magnesium and aluminum salts of phosphoric, carbonic, citric or other suitable, weak, inorganic or organic acids; or suitable organic bases, including basic amino acids; and salts or combinations thereof.

A pH-dependent coating serves to release the drug in desired areas of the gastrointestinal (GI) tract, e.g., the stomach or small intestine. When a pH-independent coating is desired, the coating is designed to achieve optimal release regardless of pH-changes in the environmental fluid, e.g., the GI tract. When the coating is formulated to release a compound according to this disclosure in the intestines (especially the upper small intestines), the coating is often called an “enteric coating”. A pH-dependent coating may include, but is not limited to, acrylic acid polymers and copolymers, for example polymers formed from acrylic acid, methacrylic acid, methyl acrylate, ammonio methylacrylate, ethyl acrylate, methyl methacrylate and/or ethyl methacrylate (e.g., Eudragit™); cellulosic polymers such as hydroxypropyl cellulose, hydroxyethyl cellulose, hydroxypropyl methyl cellulose, methyl cellulose, ethyl cellulose, cellulose acetate, cellulose acetate phthalate (CAP), cellulose acetate trimellitate, hydroxypropylmethyl cellulose phthalate, hydroxypropylmethyl cellulose succinate and carboxymethylcellulose sodium; shellac (purified lac); vinyl polymers and copolymers such as polyvinyl pyrrolidone, polyvinyl acetate, polyvinylacetate phthalate (PVAP), vinylacetate crotonic acid copolymer, and ethylene-vinyl acetate copolymers; zein; and salts and combinations thereof.

### III. Dosage Forms

One aspect of the disclosure encompasses dosage forms of anti-SARS-CoV-2 mAbs, the dosage form may comprise from about 1 mcg to about 300 mg. In one aspect, the dosage form may comprise from 500 mcg to about 300 mg, from about 1 mg to about 250 mg, from about 1 mg to about 200 mg, from about 1 mg to 180 mg, from about 5 mg to about 250 mg, from about 5 mg to about 200 mg, from about 5 mg to 180 mg, from about 5 mg to about 150 mg, from about 5 mg to about 100 mg, from about 5 mg to 80 mg, from about 5 mg to about 50 mg, from about 5 mg to about 40 mg, from about 5 mg to 30 mg, from about 1 mg to about 250 mg, from about 1 mg to about 200 mg, from about 1 mg to 180 mg, from about 1 mg to about 150 mg, from about 1 mg to about 100 mg, from about 1 mg to 80 mg, from about 1 mg to about 50 mg, from about 1 mg to about 40 mg, from about 1 mg to 30 mg from about 1 mg to about 20 mg, from about 1 mg to about 10 mg.

Dosage forms include those formulated for extended or slow release, and those formulated for immediate release.

Dosage forms also include those formulated for topical administration. For instance, a dosage form can be formulated as one or more of a gel, ointment, emulsion, microemulsion, solution, suspension, paste, gel, foam, spray, lotion, or cream. In one aspect, a topical administration dosage form is a transdermal patch. When the dosage form is formulated as a transdermal patch, the transdermal patch can contain from about 40 mg to about 60 mg, from about 80 mg to about 120 mg, or from about 180 mg to about 220 mg of A2-73 freebase in crystalline form.

Dosage forms can alternatively be formulated for oral administration. Dosage forms formulated for oral administration can be tablets to swallow, chew, or dissolve in water or under the tongue, capsules and chewable capsules, powders, granules, teas, drops, or liquid medications or syrups. In some aspects, the dosage form is an enteric coated oral formulation.

Dosage forms also encompass those formulated for subcutaneous and/or intramuscular injection.

### IV. Kits

The present disclosure also provides kits for use in treating coronavirus infection. Such kits can include one or more containers comprising the composition as described herein and optionally one or more of the second therapeutic agents as also described herein.

In some aspects, the kit can comprise instructions for use in accordance with any of the methods described herein. The included instructions can comprise, for example, a description of administration of the mAbs.

The label or package insert may indicate that the composition is used for the intended therapeutic utilities. Instructions may be provided for practicing any of the methods described herein.

The kits of this invention include suitable packaging. Suitable packaging includes, but is not limited to, chambers, vials, bottles, jars, flexible packaging (e.g., sealed Mylar or plastic bags), and the like. Also contemplated are packages for use in combination with a specific device, such as an inhaler, nebulizer, ventilator, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some aspects, present disclosure provides articles of manufacture comprising contents of the kits described above.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described cells and methods without departing from the scope of the invention, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

The term “about” or “approximately” used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “treat” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, prevent or affect the disorder, the symptom of the disease, or the predisposition toward the disease or disorder.

Alleviating a target disease/disorder includes delaying the development or progression of the disease, or reducing disease severity. Ameliorating a target disease/disorder includes improving the condition, halting or reversing the progression of the disease. Alleviating or ameliorating a disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

To achieve any of the intended therapeutic effects described herein, an effective amount of a composition herein may be administered to a subject in need of the treatment via a suitable route.

As used herein, “an effective amount” refers to the amount of each active agent required to confer therapeutic effect on the subject, either alone or in combination with one or more other active agents, such as one or more of the second therapeutic agents described herein. In some aspects, the therapeutic effect is reduction of the viral load. In some aspects, the therapeutic effect is alleviating one or more symptoms associated with a viral infection. Determination of whether an amount of the composition as described herein achieved the therapeutic effect can be readily ascertained by one of skill in the art using known methods and tools. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration, genetic factors and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. Empirical considerations, such as the half-life, generally will contribute to the determination of the dosage. Frequency of administration and/or route of administration may be determined and adjusted over the course of therapy, and is generally, but not necessarily, based on treatment and/or suppression and/or amelioration and/or delay of a target disease/disorder. Alternatively, sustained continuous release formulations of a composition as described herein may be appropriate. Various formulations and devices for achieving sustained release are known in the art. In some aspects, the effective amount can be a prophylactically effective amount (e.g., amount effective for inhibiting, treating, reducing the viral load, and/or reducing morbidity or mortality in a subject suffering from the viral infection in need of such an effect) to reduce the risk of having coronavirus infection.

The terms “subject,” “individual,” and “patient” are used interchangeably herein and refer to a mammal being assessed for treatment and/or being treated. Subjects may be human, but also include other mammals, e.g., a non-human primate, mouse, rat, guinea pig, rabbit, dog, cat, cow, chicken, etc. The subject may have or be suspected of having infection by a coronavirus. In some examples, the subject may have confirmed of having a coronavirus infection or COVID-19, or suspected of having a coronavirus infection by displaying symptoms or discomforts, such as elevated temperature, fever, cough, dyspnea, chills, persistent tremor, muscle pain, headache, sore throat, and loss of taste or smell (see ageusia and anosmia), or other symptoms of a viral pneumonia.

As used herein, the terms “nucleic acid” and “polynucleotide” refer to any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, a “receptor binding domain” or “RBD” (used interchangeably throughout) means a portion, region, or domain within a spike protein, such as a spike protein of a coronavirus, such as a SARS-Cov-2 variant, that is involved in the interaction between such spike protein and a cellular receptor of such spike protein, such an angiotensin converting enzyme 2 (“ACE2”) protein. In some embodiments, such an RBD corresponds to amino acids 319 through 541, inclusive, of a SARS-Cov-2 spike protein, or to the corresponding amino acids in a variant of such a SARS-Cov-2 virus (see, e.g., Huang etal., Acto Pharmacologica Sinica, Vol 41, pages 1141-1149 (2020)).

The term “mutation”, “modification”, or “variation”, or related terms, refers to a change in a nucleic acid sequence or amino acid sequence that differs from a reference nucleic acid sequence or a reference amino acid sequence, respectively. Examples of mutations includes a point mutation, insertion, deletion, amino acid substitution, inversion, rearrangement, splice, sequence fusion (e.g., gene fusion or RNA fusion), truncation, transversion, translocation, non-sense mutation, sequence repeat, single nucleotide polymorphism (SNP), or other genetic rearrangement. As a nonlimiting example, the QQAQ furin site mutation introduced into various coronavirus variants as disclosed herein comprises a series of amino acid substitutions introduced into the wild type Washington/Wuhan-Hu-1 isolate S protein furin site sequence RR.AR. at amino acid positions 682 to 685 and corresponding sites/positions found in other variants. As another nonlimiting example, PP spike protein stablilizing mutation as disclosed herein comprises, for example, a pair of amino acid substitutions for the wild type Washington/Wuhan-Hu-1 isolate S protein furin site sequence KV at amino acids 986 and 987 and corresponding sites/positions found in other variants.

The practice of the present invention will employ, unless otherwise indicated, conventional techniques of virology, molecular biology (including recombinant techniques), microbiology, cell biology, biochemistry and immunology, which are within the skill of the art. Such techniques are explained fully in the literature.

Without further elaboration, it is believed that one skilled in the art can, based on the above description, utilize the present invention to its fullest extent. The specific aspects provided herein are, therefore, to be construed as merely illustrative, and not limitative of the remainder of the disclosure in any way whatsoever. All publications cited herein are incorporated by reference for the purposes or subject matter referenced herein. Various aspects of the disclosure are discussed in detail below. While specific implementations are discussed, it should be understood that this is done for illustration purposes only. A person skilled in the relevant art will recognize that other components and configurations may be used without parting from the spirit and scope of the disclosure. It should be understood at the outset that although illustrative implementations of one or more aspects are illustrated below, the disclosed method may be implemented using any number of techniques. The disclosure should in no way be limited to the illustrative implementations, drawings, and techniques illustrated herein, but may be modified within the scope of the appended claims along with their full scope of equivalents.

### Specific Compositions and Methods of the Disclosure

Accordingly, the present disclosure relates, in particular, to the following non-limiting compositions and methods.

In a first embodiment, Embodiment 1, the present disclosure provides a monoclonal antibody comprising a human IgG1, IgG2, IgG3 or IgG4 heavy chain backbone amino acid sequence fused to at least one of (i) an amino acid sequence of a heavy chain variable region of tixagevimab, (ii) an amino acid sequence of a heavy chain variable region of cilgavimab and a human kappa or lambda light chain backbone fused to at least one of (iii) an amino acid sequence of a light chain variable region of tixagevimab, or (iv) an amino acid sequence of a light chain variable region of cilgavimab; wherein (i)-(iv) comprises an Fc receptor binding domain (FcγR domain).

In a second embodiment, Embodiment 2, the present disclosure provides a monoclonal antibody according to Embodiment 1, wherein the human IgG backbone amino heavy chain backbone amino acid sequence has at least 70%, at least 80% or at least 90% sequence identity to the amino acid sequence of any one of SEQ ID NOS: 1-4 and/or wherein the human kappa or lambda light chain backbone amino acid sequence has at least 70%, at least 80%, or at least 90% sequence identity to the amino acid sequence of SEQ ID NO: 5 or 6.

In another embodiment, Embodiment 3, the present disclosure provides a monoclonal antibody according to Embodiment 1, wherein the amino acid sequence of the light chain variable region of tixagevimab has at least 70%, at least 80% or at least 90% sequence identity to SEQ ID NO: 10 or 11 and/or wherein the amino acid sequence of the heavy chain variable region of tixagevimab has at least 70%, at least 80%, or at least 90% sequence identity to SEQ ID NO: 8 or 9.

In another embodiment, Embodiment 4, the present disclosure provides a monoclonal antibody according to Embodiment 1, wherein the amino acid sequence of the light chain variable region of cilgavimab has at least 70%, at least 80% or at least 90% sequence identity to SEQ ID NO: 14 or 15 and/or wherein the amino acid sequence of the heavy chain variable region of cilgavimab has at least 70%, at least 80%, or at least 90% sequence identity to SEQ ID NO: 12 or 13.

In another embodiment, Embodiment 5, the present disclosure provides a monoclonal antibody according to any one of Embodiments 1 to 4, wherein the monoclonal antibody comprises a heavy chain comprising an amino acid sequence of SEQ ID NO: 20 or 21 and a light chain comprising an amino acid sequence of SEQ ID NO: 16 or 17.

In another embodiment, Embodiment 6, the present disclosure provides a monoclonal antibody according to any one of Embodiments 1 to 4, wherein monoclonal antibody comprises a heavy chain having an amino acid sequence of SEQ ID NO: 22 or 23 and a light chain having the amino acid sequence of SEQ ID NO. 18 or 19.

In another embodiment, Embodiment 7, the present disclosure provides a monoclonal antibody according to any one of Embodiments 1 to 6, wherein the monoclonal antibody has a human-like biantennary GnGn or hybrid MGn glycoform.

In another embodiment, Embodiment 8, the present disclosure provides a nucleic acid construct encoding the monoclonal antibody according to any one of Embodiments 1 to 7, wherein the construct comprises a nucleic acid sequence having at least 70%, at least 80% or at least 90% sequence identity with any of SEQ. ID Nos. 35-41.

In another embodiment, Embodiment 9, the present disclosure provides a vector comprising the nucleic acid construct according to Embodiment 8.

In another embodiment, Embodiment 10, the present disclosure provides a transgenic Nicotiana plant comprising the monoclonal antibody according to any one of Embodiments 1 to 7, the nucleic acid construct according to Embodiment 8, and/or the vector according to Embodiment 9.

In another embodiment, Embodiment 11, the present disclosure provides a transgenic Nicotiana plant according to Embodiment 10, wherein the plant is a transgenic Nicotiana benthamiana plant.

In another embodiment, Embodiment 12, the present disclosure provides a transgenic Nicotiana plant according to Embodiment 10 or 11, having reduced expression of β 1,2-xylosyltransferase (XylT) and/or a 1,3-fucosyltransferase (FucT).

In another embodiment, Embodiment 13, the present disclosure provides a monoclonal antibody according to any one of Embodiments 1 to 7, wherein the monoclonal antibody is produced in a transgenic Nicotiana plant according to any one of Embodiments 10 to 12 having reduced expression of xylosyltransferase and/or fucosyltransferase.

In another embodiment, Embodiment 14, the present disclosure provides a pharmaceutical composition comprising (a) one or more monoclonal antibodies selected from a monoclonal antibody according to Embodiment 1, a monoclonal antibody according to Embodiment 2, a monoclonal antibody according to Embodiment 3, a monoclonal antibody according to Embodiment 4, a monoclonal antibody according to Embodiment 5, a monoclonal antibody according to Embodiment 6, a monoclonal antibody according to Embodiment 7, a monoclonal antibody according to Embodiment 13, and any combination thereof; and (b) a pharmaceutically acceptable carrier, diluent or excipient.

In another embodiment, Embodiment 15, the present disclosure provides a method of making a SARS-CoV-2 mAb variant from a parent SARS-CoV-2 mAb produced in a mammalian cell, the method comprising the steps of: (i) fusing a nucleic acid sequence encoding a heavy chain variable region of the parent SARS-CoV-2 mAb to a nucleic acid sequence encoding a heavy chain IgG backbone, and a nucleic acid sequence encoding a light chain variable region of the parent SARS-CoV-2 mAb to a nucleic acid sequence encoding a light chain backbone; (ii) restoring a nucleic acid sequence encoding an FcγR domain to the heavy chain variable region and/or the light chain variable region of the parent SARS-CoV-2 mAb to form an expression construct for expressing the SARS-CoV-2 mAb variant; (iii) introducing the expression construct into a plant cell; (iv) maintaining the plant cell for a time and under conditions sufficient for the plant to express the SARS-CoV-2 mAb variant.

In another embodiment, Embodiment 16, the present disclosure provides a method according to Embodiment 15, wherein the parent SARS-CoV-2 mAb is selected from tixagevimab and cilgavimab.

In another embodiment, Embodiment 17, the present disclosure provides a method according to Embodiment 15 or 16, wherein the plant is a N. benthamiana plant having reduced expression of xylosyltransferase and/or fucosyltransferase, and optionally is ΔXFT N. benthamiana plant.

In another embodiment, Embodiment 18, the present disclosure provides a method of treating a coronavirus infection or suspected coronavirus infection, comprising administering to a subject in need thereof an effective amount of a SARS-CoV-2 mAb variant produced in a plant cell and derived from a parent SARS-CoV-2 mAb produced in a mammalian cell.

In another embodiment, Embodiment 19, the present disclosure provides for a method according to Embodiment 18, wherein the subject has or is suspected of having an infection by a SARS virus, SARS-CoV, SARS-CoV-2, Middle East respiratory syndrome (MERS), any variant or sub-variant of any one thereof, or any combination thereof.

In another embodiment, Embodiment 20, the present disclosure provides for a method according to Embodiment 18 or 19, wherein the subject is selected from a human, a non-human primate, a dog, a cat, a cow, a pig, a sheep, a chicken, a goose and a duck.

### EXAMPLES

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Study Design

Severe acute respiratory syndrome coronavirus 2 (SARS-CoV-2) infections continue to persist around the globe, amid ongoing emergences of variants that are immune evasive. Most monoclonal antibodies (mAbs) developed as therapies for SARS-CoV-2 infection have lost utility against the Omicron variant and its subvariants. The present disclosure provides more effective mAbs, such as those expressed in non-mammalian cells. The present disclosure provides mAb cocktails to effectively combat Omicron variants, such as B.1.1.529 and BA.4/5.

### Example 2. Antibody Expression in Transgenic Plants

Tixagevimab and cilgavimab originally produced in mammalian cells were tested to be expressed in a transgenic plant. A transgenic N. benthamiana plant is used, whose transferase enzymes for constructing plant-specific xylose and fucose N-glycans were knocked down by a stable RNAi mechanism.

A glycoengineering method was used to make an mAbs construct by fusing variable regions of tixagevimab and cilgavimab onto a human IgG1 backbone, without the mutations that eliminate the Fc receptor (FcγR). Then, the gene construct was knocked into the ΔXFT N. benthamiana plants, where the transferase enzymes for constructing plant-specific endogenous β 1,2-xylosyltransferase (XylT) and/or a 1,3-fucosyltransferase (FucT) have been knocked down by a stable RNAi mechanism. Finally, transient expression of the mAbs construct was allowed in the transgenic plants.

Expression levels of plant-produced mAbs, i.e., pCilgavimab and pTixagevimab were monitored. Following expression, pCilgavimab and pTixagevimab were collected and characterized by their weight, their glycol status, and their individual neutralization against SARS-CoV-2 Omicron subvariants, such as B.1.1.529 and BA.4/5 using a pseudo virus-based neutralization assay. Finally, cocktails comprising three different amounts of pCilgavimab and pTixagevimab were tested for their combined neutralization effect against SARS-CoV-2 Omicron subvariants.

In addition, the antibody-dependent cell-mediated cytotoxicity (ADCC) of plant-produced mAb was compared to that of the same mAb produced in mammalian cells. Specifically, serial dilutions of plant-produced or mammalian cell-produced mAb (pTixagevimab) were incubated with target cells (293-SARS2-S-dfur cells, with surface-display of the spike protein of SARS-CoV-2) and ADCC bioassay reporter cells for 18-20 hours and luciferase activity was detected using Bio-Glo reagent.

### Example 3. Results

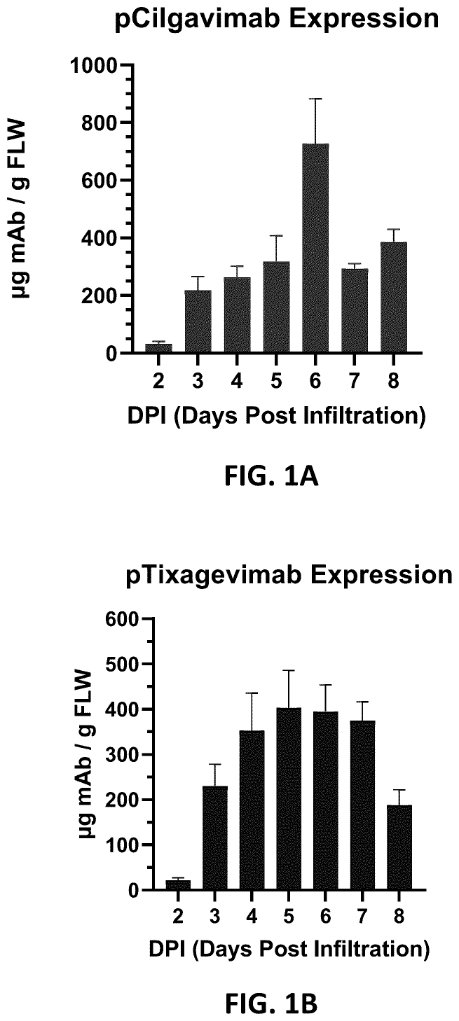

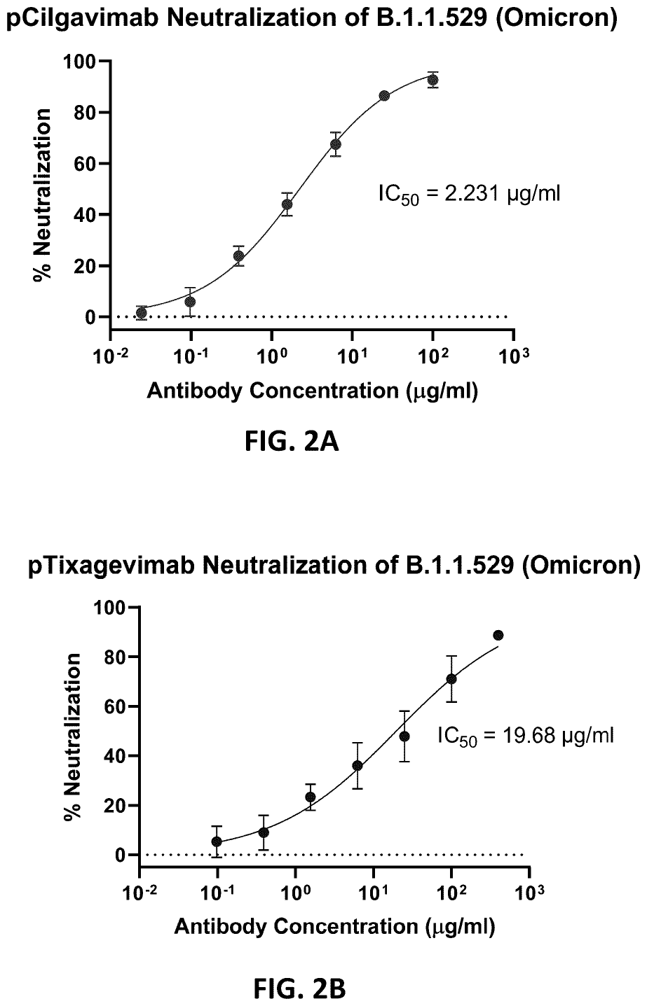

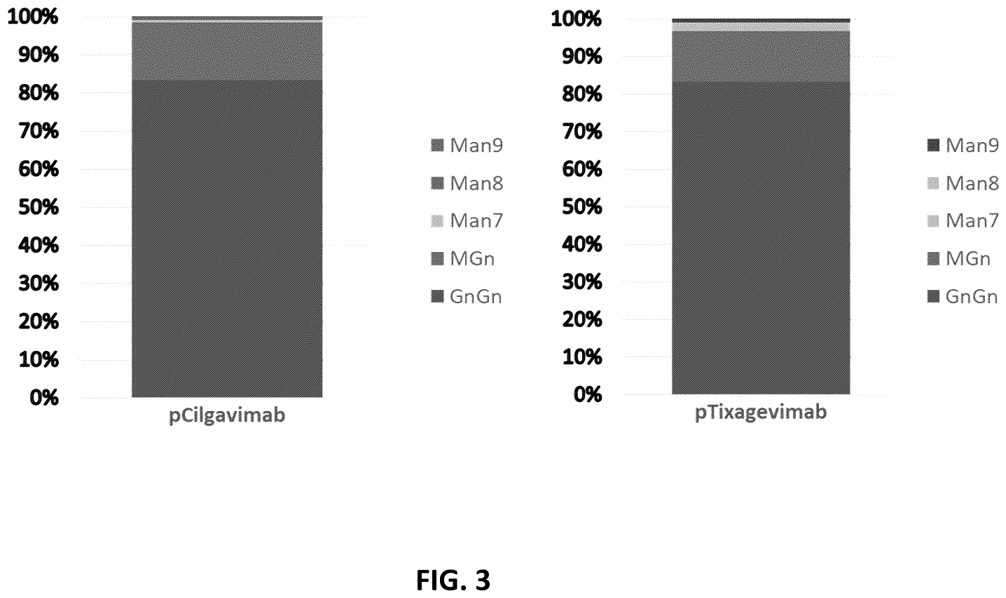

Peak expression of plant-made cilgavimab (pCilgavimab) reached approximately 726 μg of mAb/gram of fresh leaf weight (FLW) six days after agroinfiltration, while plant-made tixagevimab (pTixagevimab) expression peaked one day earlier to approximately 403 μg of mAb/g FLW (FIG. 1A-1B). Biochemical characterization of the Protein A-purified, plant-made mAbs indicated that both heavy and lights chains are expressed and form an IgG of expected size and that both mAbs have specific recognition of the RBD of SARS-CoV-2. The functionality of both pCilgavimab and pTixagevimab was indeed retained, as evidenced by their ability to neutralize the authentic parental Omicron variant (B.1.1.529) in a foci-forming assay, with an IC50 of 2.23 μg/ml and 19.68 μg/ml, respectively (FIG. 2A-2B). Furthermore, glycan analysis indicates that both pCilgavimab and pTixagevimab contain highly homogeneous N-linked glycan residues (FIG. 3), with greater than 95% of glycosylated mAbs containing human-like biantennary GnGn or a hybrid MGn glycoforms. Such results were in stark contrast to the typical heterogeneous glycan populations found on mammalian-made therapeutic proteins. Thus, our results indicated a more consistent therapeutic mAb product may be produced using ΔXFT N. benthamiana plants.

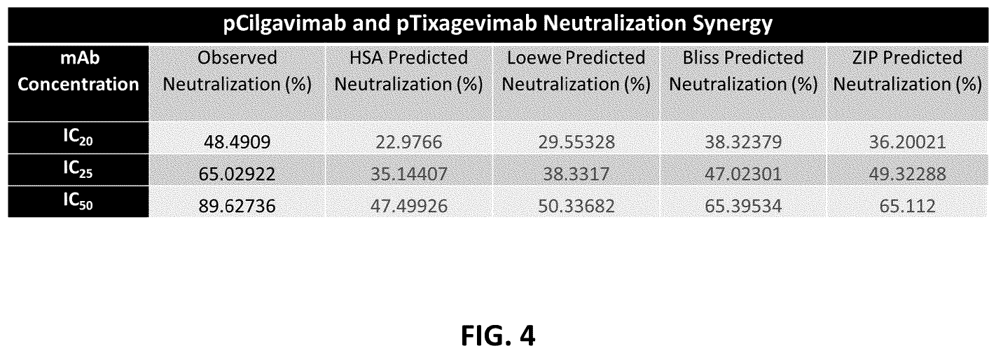

The neutralization synergy of pCilgavimab and pTixagevimab were analyzed as a dual mAb cocktail against authentic Omicron SARS-CoV-2. The mAbs were mixed, alongside individual mAb replicates at their respective IC20, IC25, and IC50 concentration and percent neutralization was empirically determined. Empirically determined percent neutralization values for individual mAbs were then analyzed in SynergyFinder.org using four models (highest single agent (HSA), Loewe, BLISS, and ZIP) to calculate a predicted percent neutralization, which represents the percent neutralization of the cocktail assuming there is no synergistic interaction between the mAbs. Notably, pCilgavimab and pTixagevimab showed synergy at all three concentrations tested (FIG. 4) as indicated by consistent higher observed neutralization values than those predicted by all four models. For example, synergy would be indicated by the Bliss and ZIP models at the IC50 concentration combination if observed percent neutralization was above 65.4% and 65.1%, respectively. Indeed, 89.6% neutralization of the pCilgavimab and pTixagevimab IC50 combination was observed, indicating synergetic neutralization was occurring against the B.1.1.529 Omicron variant.

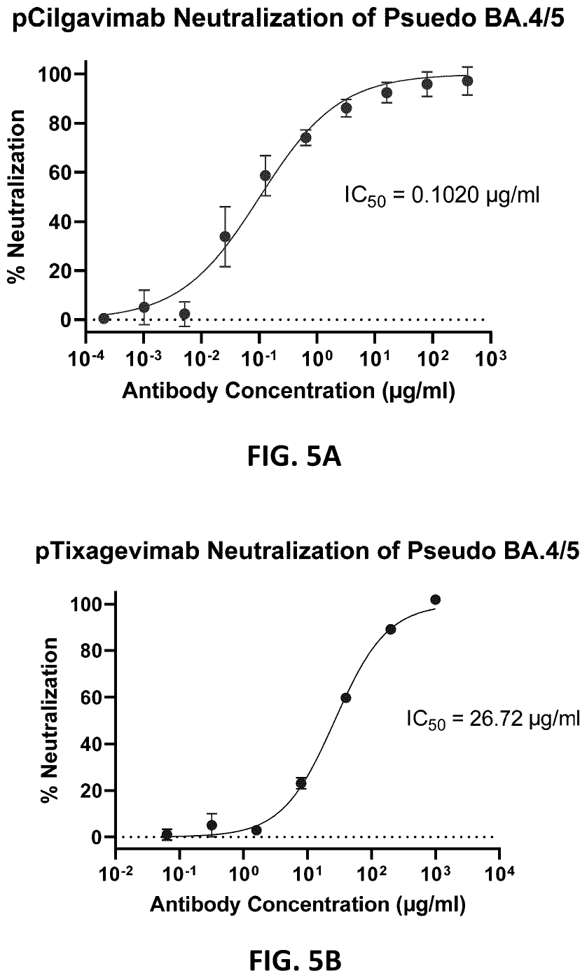

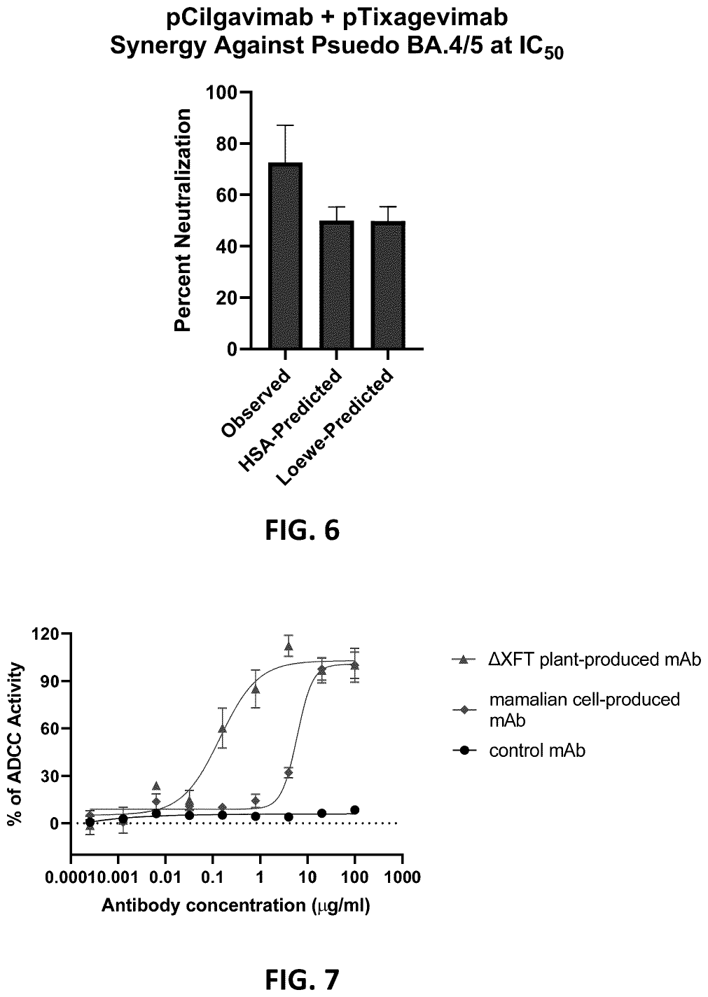

Further, pCilgavimab and pTixagevimab were tested for their neutralization against the Omicron subvariant, BA.4/5 using a pseudo virus-based neutralization assay. The test results showed that both pCilgavimab and pTixagevimab neutralized the BA.4/5 pseudo virus with IC50 values of 0.102 and 26.72 μg/ml, respectively (FIG. 5A-5B). This result was consistent with other reports of mammalian made Evusheld mAbs, where cilgavimab has increased potency against BA.4/5 pseudovirus compared to previous Omicron variants, while tixagevimab has reduced potency against BA.4/5. Furthermore, the neutralizing synergy observed between the two plant-produced mAbs against the B.1.1.529 variant carried over to the BA.4/5 variant, where the combination of mAbs at their respective IC50 concentrations resulted in ˜72.6% neutralization versus the non-synergistic reference value of ˜50% and ˜49.8% predicted by the HSA and Loewe synergy models (FIG. 6).

The ADCC activity of plant-produced mAb was compared to that of the same antibody produced in mammalian cells. The results showed that ΔXFT plant-made mAb with the GnGn glycoform exhibited much stronger ADCC activity against SARS-CoV-2 infected cells than its mammalian cell-produced counterpart (FIG. 7). These results suggest that anti-RBD mAbs made in plants have enhanced effector function against SARS-CoV-2, providing an additional mechanism of eliminating viral infection beyond neutralization and better efficacy.

### Example 4. Discussion

The present disclosure provides two plant-produced mAbs and their synergistic neutralization against Omicron variants. Both mAbs, pCilgavimab and pTixagevimab, reached high transient expression levels, extending into the hundreds of milligrams per kilogram of leaf biomass. Furthermore, both mAbs neutralize multiple SARS-CoV-2 variants, with pCilgavimab having greater potency than pTixagevimab in neutralizing both the B.1.1.529 and BA.4/5 variants. As a combination, these mAbs showed neutralizing synergy against the parental Omicron variant and remain efficacious against the BA.4/5 variant. Compared to the parent mAbs, pCilgavimab and pTixagevimab restored the FcγR binding and contained highly homogeneous, human-like GnGn glycans. Given that therapeutic mAbs against SARS-CoV-2 require Fc effector functions for optimal protection and that GnGn glycoform increases binding to certain Fc receptors on immune, but without introducing the risk of antibody-dependent enhancement of infection. These plant-made mAbs had potential to have increased therapeutic efficacy over their mammalian-made counterparts. The present disclosure set the foundation for plants to serve as an alternative platform for producing consistent, efficacious mAbs as SARS-CoV-2 therapeutics

The present disclosure made plant-produced counterparts of tixagevimab and cilgavimab, in ΔXFT Nicotiana benthamiana plants. Results showed these plant-made mAbs, pTixagevimab and pCilgavimab, efficiently neutralized multiple omicron subvariants of SARS-CoV-2 including the BA.5 with the potential of enhancing their effector function. Comparing with the current tech that antibody effector function was eliminated for the fear of side effect of antibody-dependent enhancement (ADE) of infection, the present disclosure showed the effector function was required for full function against SARS-CoV-2. Using glycoengineering, the present disclosure eliminated the risk of ADE, but restored the required effector function, and further enhanced effector function. Among other aspects, the present disclosure provides plant-produced anti-SARS-CoV-2 mAbs with restored Fc receptor binding and effector function; such mAbs exhibited the GnGn glycoform and thus enhanced ADCC activity and the GnGn glycoform posed no risk of ADE. The plant-produced pCilgavimab and pTixagevimab were effective against SARS-CoV-2 variants. In summary, pCilgavimab and pTixagevimab were able to restore effector function, but possessed little or no risk of side effect. Further showed was the increase effector function by glycoengineering, which lead to further efficacy. Further, the present disclosure was able to reduce the production cost by using plant expression. Overall, the present disclosure showed a safer, more efficacious, and more economical method of producing anti-SARS-CoV-2 mAbs.

What is claimed is:

1. A monoclonal antibody comprising a human IgG1, IgG2, IgG3 or IgG4 heavy chain backbone amino acid sequence fused to at least one of
(i) an amino acid sequence of a heavy chain variable region of tixagevimab,
(ii) an amino acid sequence of a heavy chain variable region of cilgavimab
and a human kappa or lambda light chain backbone fused to at least one of
(iii) an amino acid sequence of a light chain variable region of tixagevimab, or
(iv) an amino acid sequence of a light chain variable region of cilgavimab; wherein (i)-(iv) comprises an Fc receptor binding domain (FcγR domain).

2. The monoclonal antibody of claim 1, wherein the human IgG backbone amino heavy chain backbone amino acid sequence has at least 70%, at least 80% or at least 90% sequence identity to the amino acid sequence of any one of SEQ ID NOS: 1-4 and/or wherein the human kappa or lambda light chain backbone amino acid sequence has at least 70%, at least 80%, or at least 90% sequence identity to the amino acid sequence of SEQ ID NO: 5 or 6.

3. The monoclonal antibody of claim 1, wherein the amino acid sequence of the light chain variable region of tixagevimab has at least 70%, at least 80% or at least 90% sequence identity to SEQ ID NO: 10 or 11 and/or wherein the amino acid sequence of the heavy chain variable region of tixagevimab has at least 70%, at least 80%, or at least 90% sequence identity to SEQ ID NO: 8 or 9

4. The monoclonal antibody of claim 1, wherein the amino acid sequence of the light chain variable region of cilgavimab has at least 70%, at least 80% or at least 90% sequence identity to SEQ ID NO: 14 or 15 and/or wherein the amino acid sequence of the heavy chain variable region of cilgavimab has at least 70%, at least 80%, or at least 90% sequence identity to SEQ ID NO: 12 or 13.

5. The monoclonal antibody of claim 1, comprising a heavy chain comprising an amino acid sequence of SEQ ID NO: 20 or 21 and a light chain comprising an amino acid sequence of SEQ ID NO: 16 or 17.

6. The monoclonal antibody of claim 1, comprising a heavy chain having an amino acid sequence of SEQ ID NO: 22 or 23 and a light chain having the amino acid sequence of SEQ ID NO. 18 or 19.

7. The monoclonal antibody of claim 1, having a human-like biantennary GnGn or hybrid MGn glycoform.

8. A nucleic acid construct encoding the monoclonal antibody of claim 1, the construct comprising a nucleic acid sequence having at least 70%, at least 80% or at least 90% sequence identity with any of SEQ. ID NOs. 35-41.

9. A vector comprising the nucleic acid construct of claim 8.

10. A transgenic Nicotiana plant comprising the nucleic acid construct of claim 8.

11. The transgenic Nicotiana plant of claim 10, wherein the plant is a transgenic Nicotiana benthamiana plant.

12. The transgenic Nicotiana benthamiana plant of claim 11, having reduced expression of β 1,2-xylosyltransferase (XylT) and/or a 1,3-fucosyltransferase (FucT).

13. A monoclonal antibody produced in the transgenic Nicotiana plant of claim 10, wherein the transgenic Nicotiana plant has reduced expression of a xylosyltransferase and/or a fucosyltransferase.

14. A pharmaceutical composition comprising the monoclonal antibody of claim 13 and a pharmaceutically acceptable carrier, diluent or excipient.

15. A method of making a SARS-CoV-2 mAb variant from a parent SARS-CoV-2 mAb produced in a mammalian cell, comprising the steps of:
(i) fusing a nucleic acid sequence encoding a heavy chain variable region of the parent SARS-CoV-2 mAb to a nucleic acid sequence encoding a heavy chain IgG backbone, and a nucleic acid sequence encoding a light chain variable region of the parent SARS-CoV-2 mAb to a nucleic acid sequence encoding a light chain backbone;
(ii) restoring a nucleic acid sequence encoding an FcγR domain to the heavy chain variable region and/or the light chain variable region of the parent SARS-CoV-2 mAb to form an expression construct for expressing the SARS-CoV-2 mAb variant;
(iii) introducing the expression construct into a plant cell;
(iv) maintaining the plant cell for a time and under conditions sufficient for the plant to express the SARS-CoV-2 mAb variant.

16. The method of claim 15, wherein the parent SARS-CoV-2 mAb is selected from tixagevimab and cilgavimab.

17. The method of claim 15, wherein the plant is a Nicotiana benthamiana plant having reduced expression of xylosyltransferase and/or fucosyltransferase, and optionally is a ΔXFT N. benthamiana plant.

18. A method of treating a coronavirus infection or suspected coronavirus infection, comprising administering to a subject in need thereof an effective amount of a SARS-CoV-2 mAb variant produced in a plant cell and derived from a parent SARS-CoV-2 mAb produced in a mammalian cell.

19. The method of claim 18, wherein the subject has or is suspected of having an infection by a SARS virus, SARS-CoV, SARS-CoV-2, Middle East respiratory syndrome (MERS), any variant or sub-variant of any one thereof, or any combination thereof.

20. The method of claim 18, wherein the subject is selected from a human, a non-human primate, a dog, a cat, a cow, a pig, a sheep, a chicken, a goose and a duck.


SEQUENCE LISTING (41 sequences)

SEQ ID NO: 1 - protein, 330 aa, Homo sapiens [sequence omitted]

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 107 aa, Homo sapiens [sequence omitted]

SEQ ID NO: 6 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 19 aa, synthetic construct
MGWSCIILFLVATATGVHS

SEQ ID NO: 8 - protein, 123 aa, synthetic construct
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 9 - protein, 142 aa, synthetic construct
MGWSCIILFLVATATGVHSQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 10 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 11 - protein, 128 aa, synthetic construct
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 12 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 13 - protein, 150 aa, synthetic construct
MGWSCIILFLVATATGVHSEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 14 - protein, 112 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 15 - protein, 131 aa, synthetic construct
MGWSCIILFLVATATGVHSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 16 - protein, 216 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 235 aa, synthetic construct
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 219 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 238 aa, synthetic construct
MGWSCIILFLVATATGVHSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 453 aa, synthetic construct
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 472 aa, synthetic construct
MGWSCIILFLVATATGVHSQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 461 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 480 aa, synthetic construct
MGWSCIILFLVATATGVHSEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 369 bp, synthetic construct
  source 1..369: mol_type = other DNA
caaatgcagttggttcaatcaggtccagaagttaagaagccaggtacatcagttaaagtttcatgtaaggcatcaggattcactttcatgtcttcagctgttcagtgggttagacaagcaaggggacaaagacttgaatggattggttggattgtgataggttctggaaatacaaactatgcacaaaagtttcaagaaagagtgactattactagagatatgtcaacttctactgcttatatggaactttcttctttgagatcagaggacacagctgtttattattgcgcagcaccttattgctcatctatatcttgtaatgatggttttgacatttggggacaaggaacaatggttactgtttcttct

SEQ ID NO: 25 - DNA, 429 bp, synthetic construct
  source 1..429: mol_type = other DNA
acaatgggatggtcttgtatcatccttttcttggttgcaacagctactggtgttcattctcaaatgcagttggttcaatcaggtccagaagttaagaagccaggtacatcagttaaagtttcatgtaaggcatcaggattcactttcatgtcttcagctgttcagtgggttagacaagcaaggggacaaagacttgaatggattggttggattgtgataggttctggaaatacaaactatgcacaaaagtttcaagaaagagtgactattactagagatatgtcaacttctactgcttatatggaactttcttctttgagatcagaggacacagctgtttattattgcgcagcaccttattgctcatctatatcttgtaatgatggttttgacatttggggacaaggaacaatggttactgtttcttct

SEQ ID NO: 26 - DNA, 392 bp, synthetic construct
  source 1..392: mol_type = other DNA
gaagttcagcttgtggagtcaggtggaggtcttgttaagccaggtggatcacttagactttcttgcgctgcttcaggtttcacattcagggatgtttggatgtcatgggtgagacaggctcctggtaaaggattggagtgggttggtagaataaaatctaaaatcgatggaggtactactgattacgctgcaccagtgaagggaaggtttacaatatcaagagatgattcaaagaatacactttaccttcaaatgaactcattgaaaacagaagatactgctgtgtactactgcacaactgctggatcttattattacgacacagttggaccaggacttccagagggaaagtttgactattggggacagggaactcttgttacagtgtcttc

SEQ ID NO: 27 - DNA, 452 bp, synthetic construct
  source 1..452: mol_type = other DNA
acaatgggatggtcttgtatcatccttttcttggttgcaacagctactggtgttcattctgaagttcagcttgtggagtcaggtggaggtcttgttaagccaggtggatcacttagactttcttgcgctgcttcaggtttcacattcagggatgtttggatgtcatgggtgagacaggctcctggtaaaggattggagtgggttggtagaataaaatctaaaatcgatggaggtactactgattacgctgcaccagtgaagggaaggtttacaatatcaagagatgattcaaagaatacactttaccttcaaatgaactcattgaaaacagaagatactgctgtgtactactgcacaactgctggatcttattattacgacacagttggaccaggacttccagagggaaagtttgactattggggacagggaactcttgttacagtgtcttc

SEQ ID NO: 28 - DNA, 993 bp, synthetic construct
  source 1..993: mol_type = other DNA
gctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 29 - DNA, 327 bp, synthetic construct
  source 1..327: mol_type = other DNA
gaaatagttcttactcaatcacctggtactctttctctttcacctggtgagagagctactctttcatgtagagcatctcagtctgtttcttcttcatacttggcatggtatcagcaaaaaccaggacaagctcctagattgttgatatatggagcttcttcaagagctactggtattccagataggttctcaggttctggttcaggtactgatttcactcttactatctcaagacttgagcctgaagattttgcagtgtattactgccagcattacggatcttcaaggggttggacttttggacaaggaactaaagttgagattaaa

SEQ ID NO: 30 - DNA, 387 bp, synthetic construct
  source 1..387: mol_type = other DNA
acaatgggatggtcttgtatcatccttttcttggttgcaacagctactggtgttcattctgaaatagttcttactcaatcacctggtactctttctctttcacctggtgagagagctactctttcatgtagagcatctcagtctgtttcttcttcatacttggcatggtatcagcaaaaaccaggacaagctcctagattgttgatatatggagcttcttcaagagctactggtattccagataggttctcaggttctggttcaggtactgatttcactcttactatctcaagacttgagcctgaagattttgcagtgtattactgccagcattacggatcttcaaggggttggacttttggacaaggaactaaagttgagattaaa

SEQ ID NO: 31 - DNA, 396 bp, synthetic construct
  source 1..396: mol_type = other DNA
acaatgggatggtcttgtatcatccttttcttggttgcaacagctactggtgttcattctgatatcgtgatgactcagtctccagattctcttgcagtttcacttggagagagagcaactattaattgcaagtcatcacaatcagtgttgtattcttctaataacaagaactaccttgcttggtatcaacaaaagccaggacagcctccaaagttgttgatgtactgggcatcaacaagagagtcaggtgttccagatagattctctggttctggttcaggagctgagtttacacttacaatctcttctcttcaggctgaagatgttgcaatatattactgtcaacaatattattctactttgacttttggtggtggaactaaagttgagatcaag

SEQ ID NO: 32 - DNA, 396 bp, synthetic construct; identical to SEQ ID NO: 31
  source 1..396: mol_type = other DNA

SEQ ID NO: 33 - DNA, 324 bp, synthetic construct
  source 1..324: mol_type = other DNA
aggactgtggcagcaccatctgtgttcatattcccaccatcagatgaacaacttaagtcaggaacagcttcagttgtttgtttgcttaacaacttttacccaagggaggcaaaggttcaatggaaggtggataacgctttgcaatcaggaaactctcaggagtctgttacagaacaggattcaaaggattctacttactcattgtcttcaacattgactctttctaaggctgattacgagaagcacaaggtttatgcatgtgaagtgactcaccaaggtctttcttcaccagttactaagtcattcaatagaggagaatgctga

SEQ ID NO: 34 - DNA, 1362 bp, synthetic construct
  source 1..1362: mol_type = other DNA
caaatgcagttggttcaatcaggtccagaagttaagaagccaggtacatcagttaaagtttcatgtaaggcatcaggattcactttcatgtcttcagctgttcagtgggttagacaagcaaggggacaaagacttgaatggattggttggattgtgataggttctggaaatacaaactatgcacaaaagtttcaagaaagagtgactattactagagatatgtcaacttctactgcttatatggaactttcttctttgagatcagaggacacagctgtttattattgcgcagcaccttattgctcatctatatcttgtaatgatggttttgacatttggggacaaggaacaatggttactgtttcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 35 - DNA, 1422 bp, synthetic construct
  source 1..1422: mol_type = other DNA
acaatgggatggtcttgtatcatccttttcttggttgcaacagctactggtgttcattctcaaatgcagttggttcaatcaggtccagaagttaagaagccaggtacatcagttaaagtttcatgtaaggcatcaggattcactttcatgtcttcagctgttcagtgggttagacaagcaaggggacaaagacttgaatggattggttggattgtgataggttctggaaatacaaactatgcacaaaagtttcaagaaagagtgactattactagagatatgtcaacttctactgcttatatggaactttcttctttgagatcagaggacacagctgtttattattgcgcagcaccttattgctcatctatatcttgtaatgatggttttgacatttggggacaaggaacaatggttactgtttcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 36 - DNA, 1386 bp, synthetic construct
  source 1..1386: mol_type = other DNA
gaagttcagcttgtggagtcaggtggaggtcttgttaagccaggtggatcacttagactttcttgcgctgcttcaggtttcacattcagggatgtttggatgtcatgggtgagacaggctcctggtaaaggattggagtgggttggtagaataaaatctaaaatcgatggaggtactactgattacgctgcaccagtgaagggaaggtttacaatatcaagagatgattcaaagaatacactttaccttcaaatgaactcattgaaaacagaagatactgctgtgtactactgcacaactgctggatcttattattacgacacagttggaccaggacttccagagggaaagtttgactattggggacagggaactcttgttacagtgtcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 37 - DNA, 1446 bp, synthetic construct
  source 1..1446: mol_type = other DNA
acaatgggatggtcttgtatcatccttttcttggttgcaacagctactggtgttcattctgaagttcagcttgtggagtcaggtggaggtcttgttaagccaggtggatcacttagactttcttgcgctgcttcaggtttcacattcagggatgtttggatgtcatgggtgagacaggctcctggtaaaggattggagtgggttggtagaataaaatctaaaatcgatggaggtactactgattacgctgcaccagtgaagggaaggtttacaatatcaagagatgattcaaagaatacactttaccttcaaatgaactcattgaaaacagaagatactgctgtgtactactgcacaactgctggatcttattattacgacacagttggaccaggacttccagagggaaagtttgactattggggacagggaactcttgttacagtgtcttctgctagcaccaagggaccttctgtttttccacttgctccttcttctaagtctacttctggtggaactgctgctttgggttgtttggtgaaagattactttcctgagccagtgaccgtttcttggaactcaggtgctcttacatctggtgttcatactttcccagctgttcttcaatcttcaggactttactcactttcttctgttgttaccgttccttcttcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtgtccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaatga

SEQ ID NO: 38 - DNA, 651 bp, synthetic construct
  source 1..651: mol_type = other DNA
gaaatagttcttactcaatcacctggtactctttctctttcacctggtgagagagctactctttcatgtagagcatctcagtctgtttcttcttcatacttggcatggtatcagcaaaaaccaggacaagctcctagattgttgatatatggagcttcttcaagagctactggtattccagataggttctcaggttctggttcaggtactgatttcactcttactatctcaagacttgagcctgaagattttgcagtgtattactgccagcattacggatcttcaaggggttggacttttggacaaggaactaaagttgagattaaaaggactgtggcagcaccatctgtgttcatattcccaccatcagatgaacaacttaagtcaggaacagcttcagttgtttgtttgcttaacaacttttacccaagggaggcaaaggttcaatggaaggtggataacgctttgcaatcaggaaactctcaggagtctgttacagaacaggattcaaaggattctacttactcattgtcttcaacattgactctttctaaggctgattacgagaagcacaaggtttatgcatgtgaagtgactcaccaaggtctttcttcaccagttactaagtcattcaatagaggagaatgctga

SEQ ID NO: 39 - DNA, 711 bp, synthetic construct
  source 1..711: mol_type = other DNA
acaatgggatggtcttgtatcatccttttcttggttgcaacagctactggtgttcattctgaaatagttcttactcaatcacctggtactctttctctttcacctggtgagagagctactctttcatgtagagcatctcagtctgtttcttcttcatacttggcatggtatcagcaaaaaccaggacaagctcctagattgttgatatatggagcttcttcaagagctactggtattccagataggttctcaggttctggttcaggtactgatttcactcttactatctcaagacttgagcctgaagattttgcagtgtattactgccagcattacggatcttcaaggggttggacttttggacaaggaactaaagttgagattaaaaggactgtggcagcaccatctgtgttcatattcccaccatcagatgaacaacttaagtcaggaacagcttcagttgtttgtttgcttaacaacttttacccaagggaggcaaaggttcaatggaaggtggataacgctttgcaatcaggaaactctcaggagtctgttacagaacaggattcaaaggattctacttactcattgtcttcaacattgactctttctaaggctgattacgagaagcacaaggtttatgcatgtgaagtgactcaccaaggtctttcttcaccagttactaagtcattcaatagaggagaatgctga

SEQ ID NO: 40 - DNA, 660 bp, synthetic construct
  source 1..660: mol_type = other DNA
gatatcgtgatgactcagtctccagattctcttgcagtttcacttggagagagagcaactattaattgcaagtcatcacaatcagtgttgtattcttctaataacaagaactaccttgcttggtatcaacaaaagccaggacagcctccaaagttgttgatgtactgggcatcaacaagagagtcaggtgttccagatagattctctggttctggttcaggagctgagtttacacttacaatctcttctcttcaggctgaagatgttgcaatatattactgtcaacaatattattctactttgacttttggtggtggaactaaagttgagatcaagaggactgtggcagcaccatctgtgttcatattcccaccatcagatgaacaacttaagtcaggaacagcttcagttgtttgtttgcttaacaacttttacccaagggaggcaaaggttcaatggaaggtggataacgctttgcaatcaggaaactctcaggagtctgttacagaacaggattcaaaggattctacttactcattgtcttcaacattgactctttctaaggctgattacgagaagcacaaggtttatgcatgtgaagtgactcaccaaggtctttcttcaccagttactaagtcattcaatagaggagaatgctga

SEQ ID NO: 41 - DNA, 720 bp, synthetic construct
  source 1..720: mol_type = other DNA
acaatgggatggtcttgtatcatccttttcttggttgcaacagctactggtgttcattctgatatcgtgatgactcagtctccagattctcttgcagtttcacttggagagagagcaactattaattgcaagtcatcacaatcagtgttgtattcttctaataacaagaactaccttgcttggtatcaacaaaagccaggacagcctccaaagttgttgatgtactgggcatcaacaagagagtcaggtgttccagatagattctctggttctggttcaggagctgagtttacacttacaatctcttctcttcaggctgaagatgttgcaatatattactgtcaacaatattattctactttgacttttggtggtggaactaaagttgagatcaagaggactgtggcagcaccatctgtgttcatattcccaccatcagatgaacaacttaagtcaggaacagcttcagttgtttgtttgcttaacaacttttacccaagggaggcaaaggttcaatggaaggtggataacgctttgcaatcaggaaactctcaggagtctgttacagaacaggattcaaaggattctacttactcattgtcttcaacattgactctttctaaggctgattacgagaagcacaaggtttatgcatgtgaagtgactcaccaaggtctttcttcaccagttactaagtcattcaatagaggagaatgctga